(19) United States
(12) Patent Application Publication
(10) Pub. No.: US 2021/0402403 A1
(43) Pub. Date: Dec. 30, 2021

(54) SCREENING AND SORTING OF SINGLE CELLS

(71) Applicant: KATHOLIEKE UNIVERSITEIT LEUVEN, Leuven (BE)

(72) Inventors: Jeroen Lammertyn, Neerijse (BE); Karen VanHoorelbeke, Zwevegem (BE); Nick Geukens, Westerlo (BE); Sara Horta, Leiria (PT); Jolien Breukers, Heverlee (BE)

(21) Appl. No.: 17/291,972

(22) PCT Filed: Nov. 8, 2019
(86) PCT No.: PCT/EP2019/080702

§ 371 Date: May 6, 2021

Related U.S. Application Data
(60) Provisional application No. 62/926,337, filed on Oct. 25, 2019.

(30) Foreign Application Priority Data
Nov. 8, 2018 (GB) 1818211.3
Nov. 8, 2018 (GB) 1818215.4

(51) Int. Cl. B01L 3/00

(52) U.S. Cl. CPC B01L 3/502761; B01L 2300/0819; B01L 2200/0652; B01L 2200/027; B01L 2300/0864; B01L 2200/10; B01L 2200/16; B01L 2200/0689; B01L 2300/0893; B01L 2200/025

(57) ABSTRACT

In general the present invention concerns 1) single cell trapping of a viable cell in separate well from a plurality of wells in an array of wells, 2) single cell analysis for the selected cell and 3) single cell lifting of the yet viable cell from the well by an optical tweezer. Furthermore resent invention concerns a cell trap and lift device for B lymphocytes, the device comprising an array of wells in in polymer matrix comprising an off-stoichiometry thiol-ene polymer of the group consisting of off-stoichiometry thiol-ene (OSTE) and off-stoichiometry thiol-ene-epoxy (OSTE+) or a combination thereof that have been grafted with methacrylated polyethylene glycol (methoxy polyethylene glycol methacrylate or (M-PEG-M)) of a number average molecular weight of Mn 2000. It furthermore concerns using the B lymphocyte trap and lift device for trapping single B lymphocyte cells in wells of the device of present invention and lifting said cell from the cell trapping well by optical tweezers, preferably single beam tweezers.

### CROSS-REFERENCE TO RELATED APPLICATION

This application is a national phase entry under 35 U.S.C. § 371 of PCT International Patent Application No. PCT/EP2019/080702, filed Nov. 8, 2019, designating the United States of America, and published, in English, as PCT International Publication No. WO/2020/094848 A1 on May 14, 2020, which application claims priority to U.S. Patent Application Ser. No. 62/926,337, filed Oct. 25, 2019, Great Britain application GB20180018211 filed Nov. 8, 2018, and Great Britain application GB20180018215 filed Nov. 8, 2018.

### TECHNICAL FIELD

In general the application concerns 1) single cell trapping of a viable cell in separate well from a plurality of wells in an array of wells, 2) single cell analysis for the selected cell, and 3) single cell lifting of the yet viable cell from the well by an optical tweezer.

Furthermore the application concerns a cell trap and lift device for B lymphocytes, the device comprising an array of wells in polymer matrix composed of alternating copolymers with thiol-ene groups, for instance, a polymer matrix comprising an off-stoichiometry thiol-ene polymer of the group consisting of off-stoichiometry thiol-ene (OSTE) and off-stoichiometry thiol-ene-epoxy (OSTE+) or a combination thereof that have been grafted with methacrylated polyethylene glycol (methoxy polyethylene glycol methacrylate or (M-PEG-M)) of a number average molecular weight of Mn 2000. It furthermore concerns using the B lymphocyte trap and lift device for trapping single B lymphocyte cells in wells of the device of present invention and lifting said cell from the cell trapping well by optical tweezers, preferably single beam tweezers.

### BACKGROUND

FACS sorting and single cell sequencing, have allowed to progress in the discovery of new antibodies and therefore to improve immunoassays for disease diagnostics. However, faster screening tools and higher efficiency rates are required and efficient sorting systems for n single B cells.

Disclosed herein is a system for identification and sorting of viable individual cells in a high-throughput fashion.

### BRIEF SUMMARY

The described system solves the problems of the related art by of manipulating (human) B cells for single B cell selection without losing its viability.

Described herein is the integration of optical tweezers with a PEG-grafted OSTE+ microwell array, this array being grafted with methacrylated polyethylene glycol (methoxy polyethylene glycol methacrylate or (M-PEG-M)) of a number average molecular weight of around Mn 2000, for instance 1800-2200 and preferably 1950-2050. This system retrieves single cells out of microwells for high-throughput screening of single cell responses to delivered reagents and allows collecting cells with a positive signal for further analysis.

Further scope of applicability of the disclosure will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

Described is a cell sorting device that comprises 1) a microarray [1] of microwells [e.g., 1a], 2) an elongate or oblong conduit [14], the microwell array [1] being comprised in the elongate or oblong conduit [14], and 3) channels, characterized in that the microwell array [1] is positioned between a) at one side the upstream part of the conduit [7] with at least one inlet port [11] of the conduit [14] and b) at another side the downstream part [10] of the conduit [14] with at least one outlet port [4] of the conduit [14] and further characterized in that the microwell microarray [1] is being positioned between the ports or apertures (e.g., [8] or [28]) of i) a first set of branching channels [12] and ii) a second set of branching channels [13], and wherein these two sets of branching channels are outside the conduit [14] but connect with or are engaged with the conduit [14] via ports or apertures (e.g., [8] or [28]) in the conduit [14]. In an embodiment the present invention also provides that each fluid channel set ([12] & [13]) comprises a fluid inlet ([3] & fluid outlet [6]). In one embodiment, also provided is that the conduit [14] is a conduit for an aqueous liquid and the branched channels [12] and [13] are channels for an aqueous liquid. In a further embodiment, the cell sorting device according to any one of the above embodiments also provides that microwells are organized in parallel arrays (e.g., [1b]) of microwells or rows of microwells, which are arrays or rows separated with parallel partitions or spaces (e.g., [1c]) and which are positioned longitudinal between a ports or apertures (e.g., [8]) of the first set of branching channels and a ports or apertures (e.g., [28]) of the second set of branching channels or that the microwells which are organized in parallel arrays (e.g., [1b]) of microwells or rows of microwells which arrays or rows are separated with parallel partitions or spaces (e.g., [1c]) and which are positioned crosswise the elongate or oblong conduit [14] in an intermediate zone between the upstream part [7] and the downstream part [10] of the conduit [14]. In yet a further embodiment, the cell sorting device according to any one of the above embodiments also provides that in the first set of branching channels each channel [2] that extends from the fluid inlet port [3] branch out in additional channels [2a] which again branch out in additional channels [2b] and which at the distal end connect with or are engaged with the conduit [14] via ports or apertures (e.g., [8]) and wherein in the second set of branching channels each channel that extends from the fluid outlet port [6] branch out in additional channels which branch out in additional channels and which at the distal end connect with or are engaged with the conduit [14] via ports or apertures [e.g., 28]).

This disclosure accordingly provides the advantage of a uniform flow velocity distribution over the array of wells. The velocity increase in branched side channels as compared to the array ensures that cells do not dislodge from the micro wells but can be transported to the outlet. This helps one to work without contamination of unwanted cells and to minimize interaction of the optical tweezers with the cell. Moreover it limits interaction times with optical tweezers and it allows to find the port easily so minimizes interaction time with optical tweezers. The cells are prevented from entering in the branched fluid channels so ensures that there is no contamination of unwanted cells and there will be no loss of cells in dead volumes of the device or in tubing, collection of single cell in cell by cell manner is possible, in low volumes.

In a further embodiment, this cell sorting device according to here above described is a microfluidic cell sorting device.

In an embodiment of the cell sorting device, there is a space [24 & 23] between the microarray of microwells [1] and each zone of the wall of the conduit [14] where the branching channels are connect with or are engaged with the conduit [14] via ports or apertures (e.g., [8] or [28]), the cell sorting device being furthermore characterized in that in the upstream part [7] of the conduit [14] it comprises a first fluid inlet port [15] more distal from said the microarray of microwells [1] and a second fluid inlet port [11] more proximate to the microarray of microwells [1], wherein the first fluid inlet port [15] connects with or is engaged with two fluid channels [16] which each extend lateral and longitudinal with a space [24 or 23] and open approximate to the space [24 & 23] so to create when operational a lateral flow of a sheath fluid that sandwiches a core fluid and wherein the second fluid inlet port [11] opens more in the core of the upstream part [7] of the conduit [14] so that when operational it creates a core fluid stream towards the microarray of microwells [1].

In yet an embodiment of the cell sorting device, it comprises a solid object [26] in the downstream part [10] of the conduit [14] with a space between its rim and part of the wall of the conduit [14] so to form the channels [16] extending from the first fluid inlet port [15] and wherein the second fluid inlet port [11] opens in a cavity [27] formed by recess in the edge of solid object [26] which is faced to the microarray of microwells [1] so that when operational a core fluid stream with cells is released in said cavity [27] towards the microarray of microwells [1] and by lateral flow of a sheath fluid that sandwiches a core fluid is directed onto the microarray of microwells [1]. This solid object [26] can be a Y shaped solid plate. Moreover the second set of branching channels [13] at its end distal from the conduit [14] can be connected with or engaged with a water-in-oil droplets generator. In one embodiment, the a second set of branching channels [13] at its end distal from the conduit [14] connects with or is engaged with a fluid channel [21] that opens approximately to the outlet of an oil channel [18] so that when operational a flow with aqueous fluid comprising cells is delivered into a flow of oil from an oil fluid channel [18] to form water-in-oil droplets in an reservoir or chamber [20] with hydrophobic internal walls. This droplet based cell retrieval from sample outlet channel is advantageous in that retrieved cells can be isolated in very small volumes, much smaller than by using the other devices.

In a further embodiment, the microwell array [1] in the conduit [14] is positioned in a plane with two opposing fluid channels sets ([12] & [13]), or the microwell array [1] is a microwell array plate that at its edge site is aligned between two opposing fluid channels sets ([12] & [13]) and between the two opposing fluid conduits ports ([11] & [4]). In a further embodiment of the invention, an optical tweezer [5] is positioned under the plane or under the bottom of the microarray of microwells [1], the optical tweezer [5] mouth can be directed towards the bottom of the microarray of microwells [1] and the optical tweezer [5] when operational sends a light beam, preferably perpendicular, through the plane of the microarray of microwells [1].

In a further embodiment, each fluid channel set comprises a fluid inlet ([3] & fluid outlet [6]) and from there on the fluid channel branches out into at least 2 channels which can branches out in other at least 2 channels wherein the end channels each are engaged with a port (e.g., [8] in the conduit [14].

In a further embodiment, at least one fluid inlet [11] is at one distal end of the elongated conduit [14] opposing the at least one fluid outlet [4] at the other distal end of the elongated conduit [14].

In a particular embodiment, the elongate or oblong conduit is an enclosure or the elongate or oblong conduit is a sleeve or the elongate or oblong conduit is a groove or the elongate or oblong conduit is a liquid passage.

In yet another particular embodiment, the channel port is an aperture and/or the inlet is an aperture.

In another aspect, the disclosure provides that the microwell array [1] is for single cell per well trapping and lifting of viable cell and is characterized in that the wells are in a matrix having a thiol polymer with methoxy polyethylene glycol methacrylate chains at the surface, this chains having a number average molecular weight of a Mn value in a range between 1,500 and 2,500, preferably between 1,900 and 2,100 and most preferably 2,000.

In yet another aspect, provided is a microwell array [1] for single cell per well trapping and lifting of viable cell, characterized in that the wells are in a matrix consisting essentially of a thiol polymer with methoxy polyethylene glycol methacrylate chains at the surface, this chains having a number average molecular weight of a Mn value in a range between 1,500 and 2,500, preferably between 1,900 and 2,100 and most preferably 2,000.

In yet another aspect, the disclosure provides that the microwell array [1] is for single cell per well trapping and lifting of viable (human) B cell and is characterized in that the wells are in a matrix having a thiol polymer with methoxy polyethylene glycol methacrylate chains at the surface, this chains having a number average molecular weight of a Mn value in a range between 1,500 and 2,500, preferably between 1,900 and 2,100 and most preferably 2,000.

In yet another aspect, the disclosure provides that the microwell array [1] is for single cell per well trapping and lifting of viable (human) B cell, characterized in that the wells are in a matrix consisting essentially of a thiol polymer with methoxy polyethylene glycol methacrylate chains at the surface, this chains having a number average molecular weight of a Mn value in a range between 1,500 and 2,500, preferably between 1,900 and 2,100, and most preferably 2,000.

These microwells preferably have a diameter of a value in the range between 9-14 μm (12 μm), preferably between 10-13 μm and a depth of a value in the range between 9-14 μm (12 μm), preferably between 10-13 μm.

In yet another aspect, the disclosure provides that the methoxy polyethylene glycol methacrylate chains at the surface of said thiol polymer matrix are being bound with the thiol polymer matrix with at least one end of the methoxy polyethylene glycol methacrylate chain with a sulphur atom-containing group there between. The thiol polymer can be a thiol/ene polymer.

In yet another aspect, the disclosure provides that the polymer matrix comprises a polymer derived from a thiol terminated polymer wherein thiol groups reacted with a methoxy polyethylene glycol methacrylate. The polymer matrix can comprise oxiranyl group on its surface. Moreover the polymer matrix can comprise a thiol-ene polymer of the group consisting of off-stoichiometry thiol-enes polymer and off-stoichiometry thiol-ene-epoxies polymer.

In yet another aspect, the disclosure provides that the microwell array is comprised in an apparatus for trapping of viable single human B cell each in a well and selective lifting viable single human B cell for from it well without affecting viability, the apparatus comprising 1) a single beam optical tweezer in the 900-1200 range wave length of and with a laser power of a value between 400 mW and 600 mW or that the microwell array is comprised in an apparatus for trapping of viable single human B cell each in a well and selective lifting viable single human B cell for from it well without affecting viability, the apparatus comprising 1) a single beam optical tweezer in the 1000-1210 range wave length of and with a laser power of a value between 450 mW and 550 mW.

A further aspect is also, the use of the cell sorting device described herein for manipulation viable single cells will save guarding the viability, the manipulation comprising single cell in single well trapping, single cell analysis for the selected cell, identification B cells expressing a selected protein, optical trapping and lifting said selected cell by the optical tweezer for further manipulating of said viable cell, the use thereof for bidirectional flow or the use thereof for cell seeding, washing of non-seeded cells and delivery of reagents for the identification of the cell.

The size of the microwells (width-depth) of a certain aspect hereof is advantageously providing that B-cells can be seeded as single cells in the wells in a way that they can be analyzed and still retrieved from the wells. The size of the mixture of PEG500/2000 of a certain aspect of the invention is advantageous providing that the availability of the different functional groups on both PEGs leads to both good PDMS binding and the right hydrophilicity in order to keep the cells in motion and not adhering to the surface so that they can be lifted. The branched sample outlet of a certain aspect of the invention is advantageous providing that cell retrieval does not lead to additional cell contamination and that the process will be faster as sample outlets are closer to the microwell holes. The droplet based cell retrieval from sample outlet channel of a certain aspect of the invention is advantageous providing that retrieved cells can be isolated in very small volumes, much smaller than by using the other devices. The branched channels of a certain aspect of the invention are advantageous providing a uniform flow velocity distribution over array. It is providing a velocity increase in branched side channels as compared to the array ensures that cells do not dislodge from the microwells but can be transported to the outlet. This helps us to work without contamination of unwanted cells and to minimize interaction times of the optical tweezers with the cell.

The grouping of array of a certain aspect of the disclosure is advantageous providing that it limited interaction times with optical tweezers.

The port of branched channels in ‘funnel shape’ of a certain aspect of the invention is advantageous that it allows to find the port easily so minimizes interaction time with optical tweezers.

The extra buffer inlet for sheath flow [15,16] of a certain aspect of the disclosure is advantageously providing that cells are prevented from entering in the branched fluid channels to ensure that there is no contamination of unwanted cells.

The cell retrieval by droplets of a certain aspect of the disclosure is advantageous providing that there is no loss of cells in dead volumes of the device or in tubing, collection of single cell in cell by cell manner is possible, in low volumes.

Some embodiments are set forth directly below:
1) A microwell array (or microtray) for single cell per well trapping and lifting of viable (human) B cell, characterized in that the wells are in a matrix having a thiol polymer with methoxy polyethylene glycol methacrylate chains at the surface, this chains having have a number average molecular weight of a Mn value in a range between 1,500 and 2,500, preferably between 1,900 and 2,100, and most preferably 2,000.
2) A microwell array (or microtray) for single cell per well trapping and lifting of viable (human) B cell, characterized in that the wells are in a matrix consisting essentially of a thiol polymer with methoxy polyethylene glycol methacrylate chains at the surface, this chains having have a number average molecular weight of a Mn value in a range between 1,500 and 2,500, preferably between 1,900 and 2,100, and most preferably 2,000.
3) The microwell array according to any one of the embodiments 1 to 2, wherein the microwells have a diameter of a value in the range between 9-14 μm (12 μm), preferably between 10-13 μm and a depth of a value in the range between 9-14 μm (12 μm), preferably between 10-13 μm.
4) The microwell array according to any one of the embodiments 1 to 3, wherein methoxy polyethylene glycol methacrylate chains at the surface of said thiol polymer matrix are being bound with the thiol polymer matrix with at least one end of the methoxy polyethylene glycol methacrylate chain with a sulfur atom-containing group there between.
5) The microwell array according to any one of the embodiments 1 to 4, wherein the thiol polymer is a thiol/ene polymer.
6) The microwell array according to any one of the embodiments 1 to 5, wherein polymer matrix comprises a polymer derived from a thiol terminated polymer wherein thiol groups reacts with a methoxy polyethylene glycol methacrylate.
7) The microwell array according to any one of the embodiments 1 to 6, wherein the polymer matrix also comprises oxiranyl group on its surface.
8) The microwell array according to any one of the embodiments 1 to 6, wherein the polymer matrix comprises a thiol-ene polymer of the group consisting of off-stoichiometry thiol-enes polymer and off-stoichiometry thiol-ene-epoxies polymer.
9) The microwell array according to any one of the embodiments 1 to 8, wherein the microwell array is comprised in an apparatus for trapping of viable single human B cell each in a well and selective lifting viable single human B cell for from its well without affecting viability, the apparatus comprising 1) a single beam optical tweezer in the 900-1200 range wave length of and with a laser power of a value between 400 mW and 600 mW. 700 nanometers (nm) to 1 millimeter (mm).
10) The microwell array according to any one of the embodiments 1 to 8, wherein the microwell array is comprised in an apparatus for trapping of viable single human B cell each in a well and selective lifting of a viable single human B cell from a well without affecting viability, the apparatus comprising 1) a single beam optical tweezer in the 1000-1210 range wave length of and with a laser power of a value between 450 mW and 550 mW.
11) The use of the apparatus according to any one of the embodiments 9 to 10, for manipulation viable single human B cells will save guarding the viability, the manipulation comprising single cell in single well trapping, single cell analysis for the selected cell, identification B cells with a selected membrane bound immunoglobulin, optical trapping and lifting said selected cell by the optical tweezer for further manipulating of said viable cell.
12) The specific design of the microfluidic apparatus that can be used. The design consists of a bidirectional flow. The horizontal channels will be used for cell seeding, washing of non-seeded cells and delivery of reagents for the identification of the cell. The vertical channels will remain completely clear of cells. This vertical direction is used for transport of the tweezed cell.

Particular and preferred aspects of the invention are set out in the accompanying independent and dependent claims. Features from the dependent claims may be combined with features of the independent claims and with features of other dependent claims as appropriate and not merely as explicitly set out in the claims.

### BRIEF DESCRIPTION OF THE DRAWINGS

The disclosure will become more fully understood from the detailed description given herein below and the accompanying drawings which are given by way of illustration only, and thus are not limitative of the present invention, and wherein:

FIG. 1 is a size distribution of human B cells isolated from PBMCs. Three samples from different healthy donors were tested and human B cells isolated. Cells were imaged under the microscope and size calculated using cell profile software. Size in μm scale (n=3).

FIG. 2 is a schematic showing the fabrication of OSTE+ microwell arrays, similar as described in [6. Decrop, D. et al. ACS Appl. Mater. Interfaces 9, 10418-10426 (2017).]. First, photoresist S1818 or AZ6632 are spin coated on a silicon wafer and photopatterned with circles corresponding the desired microwell diameter. Then, Deep Reactive Ion Etch (“DRIE”) is used to obtain microwell with the desired microwell depth. In a next step, PDMS in 5:1 ratio for base:curing agent is poured on the silicon wafer and cured in an oven overnight at 60° C. Then, the PDMS is peeled off of the silicon wafer. This is the ‘PDMS stamp,’ which has pillars that correspond do the desired microwell depth and diameter. Then, a glass slide is silanized by submerging for 10 minutes in a 5% solution of 3-(Trimethoxysilyl)propyl methacrylate in methanol followed by 10 min baking at 110° C. Then, OSTE+ is spin coated on the glass slide for 30 seconds at 2500 rpm. Next, the PDMS stamp is pushed on the OSTE+ film on the glass slide. Air bubbles between the PDMS and the OSTE+ are pushed out by pressing the PDMS with a miniature rolling pin. After 5 min, the OSTE+ with PDMS stamp on top is exposed to UV at 12 mW/cm² for 2 minutes. Then, the PDMS stamp is peeled off from the OSTE+. The thermal cure can be done overnight at room temperature or for at least 2 hours in an oven at 60° C.

FIG. 3 is a graphic representation of seeding efficiency of human B cells in microwell arrays with different well diameter (ø) and depth ( [symbol] ). B cells isolated from human PBMCs were stained with DAPI and seeded in microwell arrays. After 30 minutes incubation, the arrays were washed, and images captured with Nikon fluorescent microscope in bright field and 400 nm laser. Number of well and respective presence of single or more cells per well was registered. Legend: gray bars ( [symbol] ) indicate the percentage of single cells per well; white bars ( [symbol] ) indicate the presence of two or more cells per array. Size in μm scale (n=2).

FIG. 4 is a set of two complementary pictures with B cells seeded in microwell arrays of 12±1 μm diameter and 14±1 μm depth. A) DNA stained B cells seeded in microwell array. Each blue spot corresponds to a single cell; B) Bright field image of microwell array with seeded B cells.

FIG. 5 is a graphic showing the integration of a microwell array with an optical tweezers to lift single B cells from a microwell.

FIG. 6 displays grafting of the OSTE+ microwell surfaces with methacrylated PEG (Poly(ethylene glycol) methacrylate with average Mn 360 (PEGMA) using UV thiol-ene click chemistry.

FIG. 7 A) demonstrates static contact angle measurements for OSTE+ surface without treatment, OSTE+ grafted with PEGMA Mn 360 and OSTE+ grafted with M-PEG-M Mn 2,000. Legend: white bar correspond to measurements for OSTE+ surface without treatment ( [symbol] ); white bar with black stripes correspond to OSTE+ grafted with PEGMA Mn 360 ( [symbol] ); black bar correspond to OSTE+ grafted with M-PEG-M Mn 2,000 ( [symbol] ). B) demonstrates static contact angle measurements of OSTE+ surface, and OSTE+ after submersion in the PEGMA Mn 360 solution and OSTE+ after submersion in the M-PEG-M Mn 2,000 solution without UV exposure. For each condition, 3 samples were fabricated. For each sample, 3 static contact angle measurements were performed. For each sample, the average of these 3 measurements represents the measurement of that sample, thus n=3. Statistical significance was obtained using t tests. Legend: white bar correspond to measurements for OSTE+ surface ( [symbol] ); white bar with black stripes correspond to OSTE+ after submersion in the PEGMA Mn 360 solution ( [symbol] ); black bar correspond to OSTE+ after submersion in the M-PEG-M Mn 2,000 solution without UV exposure ( [symbol] ).

FIG. 8 corresponds to study of Brownian motion (BM) of B cells seeded in 11±1 μm well after 2 hours incubation. B cells isolated from human PBMCs were seeded in microwell arrays with different treatments: without PEG treatment (control), with PEGMAM-360 and with M-PEG-M 2,000. After 2 hours incubation, videos were recorded with fluorescent microscope. Random movement of a single cell for more than 10 seconds was taken as a BM. By applying t-test, statistical significance was obtained when comparing the BM of cells seeded in control and M-PEG-M 2,000, with p value<0.05; and when comparing the BM of cells seeded in PEGMA-360 and M-PEG-M 2,000, with p value<0.001. Experiments performed in triplicates (n=3). Legend: white bar correspond to BM of cells seeded in microwell arrays without treatment ( [symbol] ); white bar with black stripes correspond to BM of cells seeded in microwell arrays with PEGMA-360 ( [symbol] ); black bar correspond to BM of cells seeded in microwell arrays with M-PEG-M 2,000 ( [symbol] ).

FIG. 9 is a graphic showing the percentage of B cells lifted from 11±1 μm depth well using optical tweezers. B cells isolated from human PBMCs were seeded in microwell arrays with different treatments: without PEG treatment (control), with PEGMA-360 and with M-PEG-M 2,000. Optical tweezers were applied in B cells, seeded in microwell, which demonstrated BM. An attempt to trap and lifting a single cell using the laser was made. In case of success, the attempt was registered as (1); in case the cell could not be lifted out of the well, the attempt was registered as (0). By applying t-test, statistical significance was obtained when comparing the lifting success of cells seeded in control and M-PEG-M 2,000, with p value<0.001; when comparing the lifting success of cells seeded in PEGMAM-360 and M-PEG-M 2,000, with p value<0.05. T test was applied on described data. Experiments performed in triplicates (n=3). Legend: white bar correspond to BM of cells seeded in microwell arrays without treatment ( [symbol] ); white bar with black stripes correspond to BM of cells seeded in microwell arrays with PEGMAM-360 ( [symbol] ); black bar correspond to BM of cells seeded in microwell arrays with M-PEG-M 2,000 ( [symbol] ).

FIG. 10 displays B cell viability after 10 mins exposition to an optical tweezer laser. A) B cells in cover slide before applying optical tweezer laser. Cells were resuspended in medium with 7-AAD dye. This dye only penetrates compromised cell membranes and labels the cells of red when excited at 550 nm. The B cell at which the laser was applied is indicated with arrow ( [symbol] ) B cells in cover slide after applying optical tweezer laser. The absence of red staining in the cell in analysis indicates that membrane integrity was maintained after 10 mins exposition to optical tweezer laser. The B cell at which the laser was applied is indicated with arrow ( [symbol] ).

FIG. 11 is a graphic showing a PDMS channel on top of the OSTE+ microwell array that is used for seeding of single cells and can be used for controlled reagent delivery. The platform is integrated with an optical tweezers set-up to elevate single cells with the response of interest out of a microwell.

FIG. 12 shows the top view of the PDMS channel from the schematic in FIG. 11 with its dimensions. The total volume of the channel is proximately 20±1 μL.

FIG. 13 corresponds to a set of microscope images showing a comparison of washing efficiency in a channel for non-treated OSTE+, OSTE+ grafted with PEGMAM-360 and OSTE+ grafted with M-PEG-M 2,000. B cells were isolated from human PBMCs and stained with commercial kit celltracker. After B cells incubation and washing step in microfluidic channel, images of the array were captured in both bright field and fluorescence.

FIG. 14 is a schematic representation of the ELISA based assay for the identification of specific B cells. B cells isolated from human PBMCs are blocked with 1% BSA and incubated with biotinylated anti-human IgG followed by streptavidin-β-galactosidase. Labeled B cells are seeded in microwell arrays and substrate (Fluorescein di(B-D-galactopyranoside)) is added. The microwell arrays are sealed with oil (FC-40). In case a cell expressing IgG on surface is seeded in the well, it becomes green due to substrate cleavage by β-galactosidase.

FIG. 15 demonstrates preliminary data obtained in ELISA based assay for the identification of IgG B cells. A) Microwell arrays with streptavidin-β-galactosidase labeled B cells. The green fluorescence was obtained with arrays were sealed with FC-40 oil. The fluorescence corresponds to the presence of a B cell with IgG on membrane. B) Measurement of labeled B cells and respective controls in spectrophotometer. 1×106 cells were added to 96 well and 1 μg/ml of substrate added before the measurement. Legend: The filled line ( [symbol] ) corresponds to B cells labeled with Biotinylated anti-IgG antibody and streptavidin-β-galactosidase. The striped line ( [symbol] ) corresponds to B cells without biotinylated anti-IgG antibody but with streptavidin-β-galactosidase. The dashed line ( [symbol] ) corresponds to B cells medium.

FIG. 16 corresponds to study of Brownian motion (BM) of B cells seeded in 11±1 μm well after 2 hours of incubation. B cells isolated from human PBMCs were seeded in microwell arrays with different treatments: without PEG treatment (OSTE+), with PEGMA 360, PEGMA 500, PEG 500/2,000 and M-PEG-M 2,000. After 2 hours incubation, videos were recorded with fluorescent microscope. Random movement of a single cell for more than 10 seconds was taken as a BM. (For all graphs, the line represents the mean and the error bars show standard deviations for 3 measurements. n=3, * represents p<0.05).

FIG. 17 is a graphic showing the percentage of B cells lifted from 11±1 μm depth well using optical tweezers. B cells isolated from human PBMCs were seeded in microwell arrays with different treatments: without PEG treatment (OSTE+), with PEGMA 360, PEGMA 500, PEG 500/2,000 and M-PEG-M 2,000. Optical tweezers were applied to B cells, seeded in microwell, which demonstrated BM. An attempt to trap and lifting a single cell using the laser was made. In case of success, the attempt was registered as (1); in case the cell could not be lifted out of the well, the attempt was registered as (0). (For all graphs, the line represents the mean and the error bars show standard deviations for 3 measurements. n=3, * represents p<0.05).

FIG. 18 shows a schematic representation of the microfluidic platform for single cell seeding and retrieval with integrated optical tweezer set-up. The microwell array and optical tweezers are not drawn to scale. Each number represents an element and a detailed description can be found in the text.

FIG. 19 shows a schematic representation of the microwell array design in the microfluidic platform for single cell seeding and retrieval.

FIG. 20 shows a close-up of a the microfluidic platform to show the detailed design of the connection between the set of branched fluid channels [12],[13] and the main conduit [14] through the port [8].

FIG. 21 displays a schematic representation of fluid flow during cell seeding and washing step. Syringe pumps are connected to each inlet/outlet and the arrows represent the flow direction ( [symbol] ). The numbers represent an element of the microfluidic platform and full description can be found in the text.

FIG. 22 shows a schematic representation of fluid flow during tweezing of a single cell. Syringe pumps are connected to each inlet/outlet and the arrows ( [symbol] ) represent the flow direction. The numbers represent an element of the microfluidic platform and full description can be found in the text.

FIG. 23 represents the process of transport a cell from the microwell array to the port using optical tweezers [8] (path indicated by dotted line [symbol] ) and the transport of the cell from the port [8] to the outlet by the microfluidic flow (path indicated by dashed line [symbol] ).

FIG. 24 is a representation of different elements of microfluidic platform with integrated droplet generation for single cell seeding and retrieval. The microwell array and optical tweezers are not drawn to scale. The numbers represent an element of the microfluidic platform and full description can be found in the text.

FIG. 25 shows a schematic representation of fluid flow in the microfluidic platform with integrated droplet generation during the cell seeding step. Syringe pumps are connected to each inlet/outlet and the arrows ( [symbol] ) represent the flow direction. The numbers represent an element of the microfluidic platform and full description can be found in the text.

FIG. 26 shows a schematic representation of fluid flow in the microfluidic platform with integrated droplet generation during the washing step. Syringe pumps are connected to each inlet/outlet and the arrows ( [symbol] ) represent the flow direction. The numbers represent an element of the microfluidic platform and full description can be found in the text.

FIG. 27 shows a schematic representation of fluid flow in the microfluidic platform with integrated droplet generation during the tweezing step. Syringe pumps are connected to each inlet/outlet and the arrows ( [symbol] ) represent the flow direction. The numbers represent an element of the microfluidic platform and full description can be found in the text.

FIG. 28 shows microscopy images of the concept of identification and isolation of a single B cell. i.) The cell is identified using fluorescence microscopy; ii.) The cell is tweezed out of the microwell by the optical tweezers; iii.) The cell is tweezed to the funnel; iv.) The cell is released by the optical tweezers and taken along by the microfluidic flow. This can be seen in the image since the optical tweezers is located in the middle of the field of view but the cell already moved further in the channel.

FIG. 29 shows a microscopy image of the buffer-in-oil droplet outlet [19] to which a tubing is connected. Cells can be seen as bright white dots. Cells do not enter the tubing but sediment below the lumen of the tubing because of gravity.

FIG. 30 illustrates with microscopy images how cells are taken out of the microfluidic chip into the tubing by using droplets. In this case, multiple B cells and platelets were encapsulated into the medium droplet. At the droplet outlet, no cells can be observed below the lumen of the tubing, which is in large contrast with FIG. 29. The cells are indicated by white arrows.

FIG. 31 indicates which part of the microfluidic design should be treated with a hydrophobic coating to enable droplet formation. This is indicated by grey filling.

Tables in this application:

TABLE 1
Different ratios of PEGMA 500 and M-PEG-M 2,000

| Ratio PEGMA 500/ | | | | | |
|---|---|---|---|---|---|
| M-PEG-M 2,000 | 4 on 1 | 2 on 1 | 1 on 1 | 1 on 2 | 1 on 4 |
| w/w % PEGMA 500 | 10 | 10 | 6.25 | 3.125 | 1.5625 |
| w/w % M-PEG-M 2,000 | 20 | 40 | 50 | 50 | 50 |
| solution in H₂O | | | | | |
| w/w % UV initiator | 1 | 1 | 1 | 1 | 1 |
| w/w % ethanol | 69 | 49 | 42.75 | 45.875 | 47.4375 |

### DETAILED DESCRIPTION

The following detailed description refers to the accompanying drawings. The same reference numbers in different drawings identify the same or similar elements. Also, the following detailed description does not limit the invention. Instead, the scope of the invention is defined by the appended claims and equivalents thereof.

Several documents are cited throughout the text of this specification. Each of the documents herein (including any manufacturer's specifications, instructions etc.) are hereby incorporated by reference; however, there is no admission that any document cited is indeed prior art of the present invention.

The disclosure is described with respect to particular embodiments and with reference to certain drawings but the invention is not limited thereto but only by the claims. The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising,” used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to the devices consisting only of components A and B. It means that with respect to the instant disclosure, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Other embodiments will be apparent to those skilled in the art after consideration of the specification and practice of the invention disclosed herein.

It is intended that the specification and examples be considered as exemplary only.

Each and every claim is incorporated into the specification as an embodiment of the disclosure. Thus, the claims are part of the description and are a further description and are in addition to the preferred embodiments of the disclosure.

Each of the claims set out a particular embodiment of the disclosure.

The following terms are provided solely to aid in the understanding of the invention.

### Definitions

OSTE+ is an off-stoichiometry thiol-ene-epoxy (TEE). Off-stoichiometry thiol-ene polymer comprises off-stoichiometry thiol-enes (OSTE) and off-stoichiometry thiol-ene-epoxies (OSTE+). OSTE resins are cured via a rapid thiol-ene “Click” reaction between thiols and allyls. The thiols and allyls react in a perfectly alternating fashion and have a very high conversion rate (up to 99%); the initial off-stoichiometry of the monomers will exactly define the number off unreacted groups left after the polymerization. With the right choice of monomers very high off-stoichiometry ratios can be attained while maintaining good mechanical properties. The off-stoichiometry thiol-ene-epoxies, or OSTE+ polymers, are created in a two-step curing process where a first rapid thiol-ene reaction defines the geometric shape of the polymer while leaving an excess of thiols and all the epoxy unreacted. In a second step all the remaining thiol groups and the epoxy groups are reacted to form an inert polymer [Saharil, Journal of Micromechanics and Microengineering 23, 025021 (2013)].

To achieve excellent processing properties of TEE thermosets the reactions of Thiol-ene coupling (TEC) and thiol-epoxy coupling (TEpC) should be temporally separated and individually controlled. This can be done by inducing sequential crosslinking of the TEE network by first curing thiol-ene groups followed by thiol-epoxy groups, or vice versa [J. A. Carioscia, et al. Polymer, vol. 48, no. 6, pp. 1526-1532, 2007]. Since thiol groups are involved in both of the two curing steps proper control mechanisms for the separation of each curing stage have to be chosen. This effectively includes external control of the initiation mechanisms for each ideally orthogonal reaction type (e.g., radical TEC and base catalyzed TEpC) by careful selection of monomers and initiators. More specifically a two-component thiol-ene-epoxy is used prepared by mixing the OSTEmerX Crystal Clear (322-40) compounds A (harder) and B (base) with a mass ratio of 1.1:1. In the first step epoxy monomers added form the ternary thiol-ene-epoxy monomer systems, where the epoxy in a second step reacts with the excess of thiols creating a final polymer article that is completely inert. Throughout the document, the first reaction step in which the thiol groups and the ene groups form bonds, is named ‘UV cure’ or ‘First UV cure.’ The second reaction step in which the excess thiol groups form bonds with the epoxy groups, is called the ‘thermal cure.’

‘Grafting of polyethylene glycol (PEG) on the OSTE+ surface’ means that the OSTE+ surface is incubated with a solution containing certain percentage of PEG dissolved in ethanol, containing also a percentage of UV initiator (1-hydroxycyclohexyl phenyl ketone 99%, CAS Number 947-19-3, Linear Formula HOC6H10COC6H5, Molecular Weight 204.26). The OSTE+ in contact with the PEG solution only had a first UV cure, meaning that allyl and thiol groups have reacted, and there are still free thiol and epoxy groups on the surface. The different types of PEG described here, have a methacrylate group that will react with the free thiol on the surface when exposed to UV.

When a ‘PDMS channel,’ or ‘microfluidic channel,’ or ‘microfluidic PDMS channel,’ or ‘microfluidic channel in PDMS’ or ‘channel’ is described, this refers to a microfluidic channel in the Polydimethylsixolane polymer which is a very common technique for prototyping microfluidics in research. For this microfluidic device fabrication, the PDMS base is mixed with a curing agent and poured into a microstructured mold. Then, the PDMS is heated to have an elastomeric replica of the mold.

Herein is successfully demonstrated collection of single B lymphocyte cells, from microwells by designing and stamp-molding a polyethyleneglycol (PEG)-grafted microwell array and combining it with an optical tweezers set-up. In a particular set up the microwell array was composed of an off-stoichiometry thiol-ene polymer, more particularly an off-stoichiometry thiol-ene-epoxy. The PEG molecules coated on the microwell surface led to enhanced Brownian motion of the cells by avoiding its adhesion to surface, resulting in higher performance of the optical tweezers for single cell trapping and sorting. By integrating the microwell array with a channel in Polydimethylsiloxane (PDMS), the PEG molecules also led to efficient washing of non-seeded cells. Thus, the PEG molecules were required for the development of a high-throughput screening of single cell responses to delivered reagents and collecting cells.

Phenotypic and genetic diversity at the single cell level is often overlooked in bulk assays. Recently, platforms for single cell studies have arisen to provide relevant information of rare cell subpopulations. In particular, studies of single B cells, e.g., FACS sorting and single cell sequencing, have allowed to progress in the discovery of new antibodies and therefore to improve immunoassays for disease diagnostics. However, faster screening tools and higher efficiency rates are still required.

Single cell screening platforms such as microwell arrays could solve this technical problem, however, collecting viable cells with a positive signal for further analysis is still challenging, especially in combination with a sealed microfluidic device. Herein, an optical tweezer system was used for lifting a single cell that was first captured in microwells with suitable dimensions to trap one single cell. We performed crosslinking of poly(ethylene glycol) (PEG) polymer chains onto a surface of the off-stoichiometry thiol-ene epoxy (OSTE+) microwells (surface grafting of PEG on the off-stoichiometry thiol-ene epoxy (OSTE+) microwells) and combined with optical forces of which we found that can overcome the interaction forces between the cell and the microwell surface single cell trapped in a cell could be lifted and translocated successfully without viability loss by lysis.

The disclosure enables the identification, sorting, and analysis of individual cells in a high-throughput fashion and B cells, isolated from fresh blood or from cryopreserved human peripheral blood mononuclear cells (PBMCs), can be used for the identification of specific antibodies. In order to achieve single cell resolution, the cells are seeded individually in microwell arrays. Subsequently the ones that present specific membrane immunoglobulins are identified by means of a sandwich ELISA based assay, which results in a fluorescent signal. Next, an optical tweezers set-up is used to retrieve the positive B cells out of the microwells and transport them across the array. Using a unique microfluidic design, the selected B cells are collected in a tube or 96 well plate for further single cell sequencing.

Different combinations of microwell diameter, microwell depth and interwell distance have been tested to optimize the cell seeding. A seeding efficiency of up to 85% was achieved with a single cell seeding of up to 64%, enabling the simultaneous analysis of up to 35,000 individual cells. After cell incubation of at least 1 hour, the optical tweezers were used to elevate the B cells of interest out of the microwells. So far up to 95% of them could be retrieved from the microwells and transported across the array. Furthermore, it was experimentally confirmed that tweezing cells for 10 minutes did not induce cell lysis.

Next to the ability of fast screening and isolation of specific B cells, this versatile platform can be used in many other single cell studies that require high-throughput cell identification and collection.

### EXAMPLES

### Example 1—Successful Integration Microwells for B Cells with Optical Tweezers

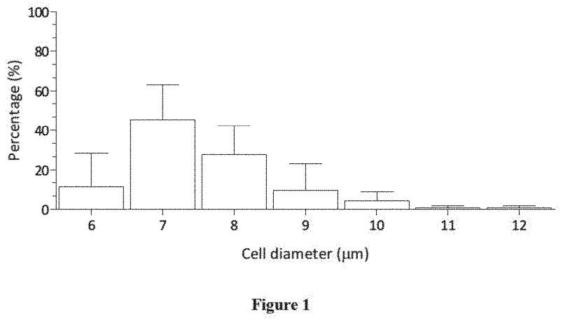

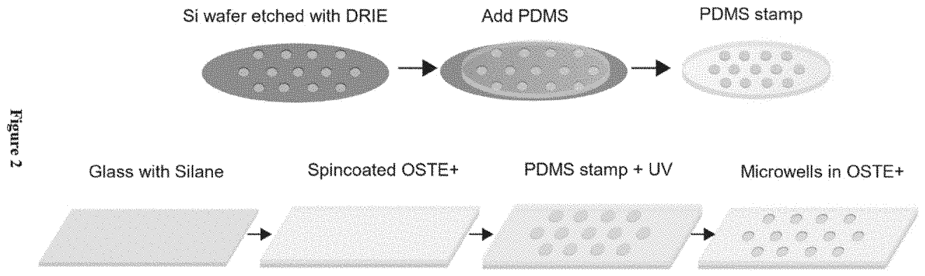

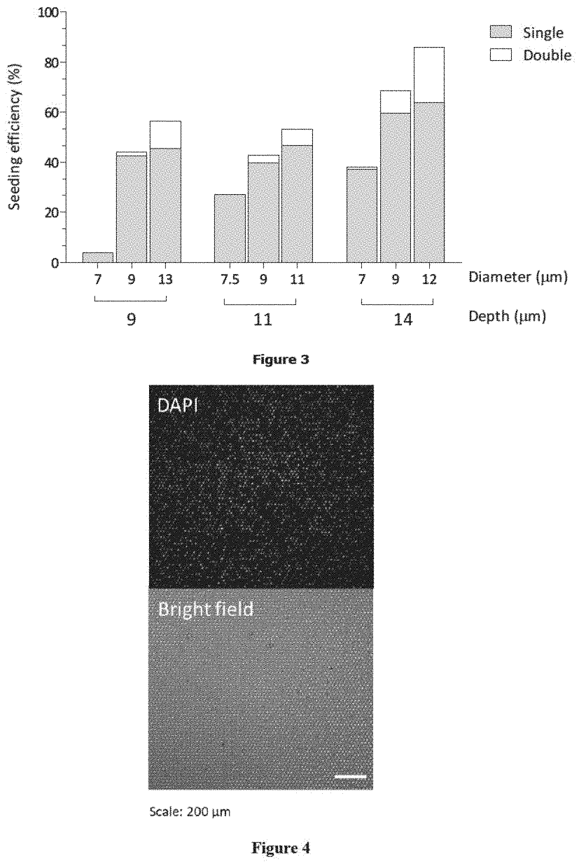

OSTE+ microwell arrays with different well diameter (ø) and depth ( [symbol] ) were designed considering the dimensions of human B cells (FIG. 1). The process of microwell array fabrication in OSTE+ using a stamp-molding technique is shown in FIG. 2. The human B cells were prepared at concentration of 107 cells/ml and stained against DNA (DAPI). Ten μl was then pipetted into the arrays and incubated for 30 mins. After incubation time, the arrays are rinsed with PBS to remove unseeded cells. Human B cell seeding efficiency was evaluated using fluorescence microscopy. Arrays with microwells of 12±1 μm diameter demonstrated a seeding of up to 80% in which 60% represent single cells. Smaller diameters lead to decreased seeding (FIG. 3). It was observed that arrays with 11±1 μm diameter and 11±1 μm depth leads to an optimal ratio of single cell seeding and low number of wells with two or more cells. Plus, preliminary experiments demonstrated that a depth of 11±1 μm is more suitable for single cell lifting when compared with 14±1 μm.

Seeding efficiency of human B cells in different microwell arrays is displayed in FIG. 4. A) DNA stained B cells seeded in microwells; B) Bright field image of microwell array with seeded B cells.

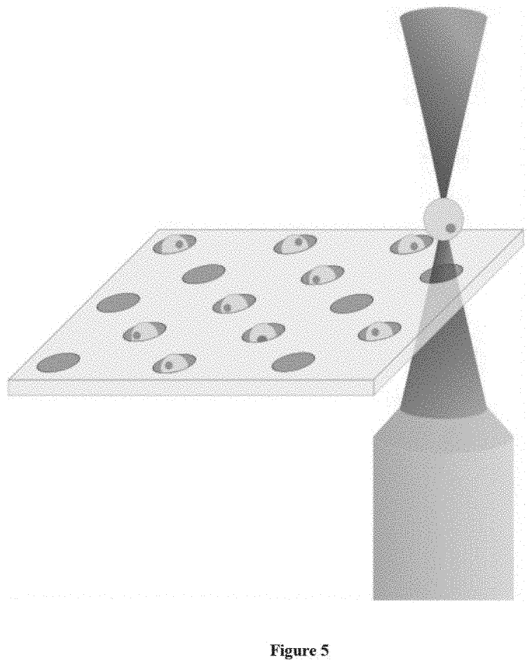

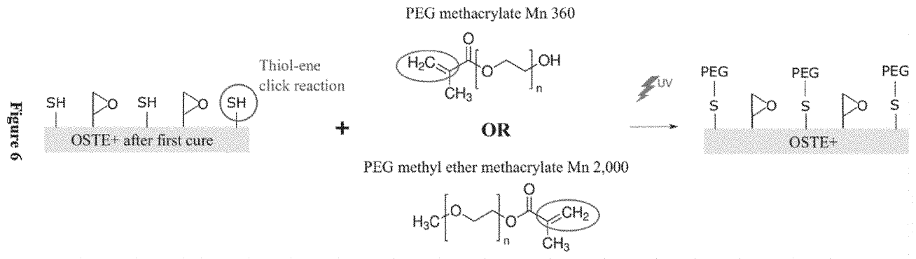

The microwell array and integration with optical tweezers is shown in FIG. 5. The optical tweezers set-up is described in [1. Decrop, D. et al. Anal. Chem. 88, 8596-8603 (2016).]. It is a 500 mW single beam optical tweezers of 1064 nm. It was seen that the optical tweezers were not efficient for lifting single B cells out of the OSTE+ microwells, because of interaction forces between the B cells and the microwell surface. To tune these interaction forces, surface grafting of two types of Poly(ethylene glycol) (PEG) methacrylate was performed on the OSTE+ surface using UV initiated thiol-ene click chemistry (FIG. 6). The first type of PEG was Poly(ethylene glycol) methacrylate with average Mn 360 (Synonym: PEGMA, CAS Number 25736-86-1, Linear Formula H2C═C(CH3)CO(OCH2CH2)nOH, MDL number MFCD00081879 and with end standing hydroxyl group as in the formula

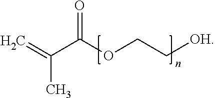

This PEGMA Mn 360 was diluted in ethanol, having 10 w/w % PEGMA, 2 w/w % UV initiator and 88 w/w % ethanol. After the first UV cure of the OSTE+ microwell array, the OSTE+ array with free thiol and epoxy groups is completely submerged in the described solution with PEGMA Mn 360. Then, this is exposed to UV of 12 mW/cm² for 5 minutes. After this, the OSTE+ array is rinsed thoroughly first with ethanol and then with water. Next, the OSTE+ array is blow dried using a N₂ gun. The thermal cure of the OSTE+ array is performed in an oven at 60° C. overnight.

The second type of PEG was Poly(ethylene glycol) methyl ether methacrylate solution average Mn 2,000, 50 wt. % in H2O (Synonym: M-PEG-M, CAS Number 26915-72-0, Linear Formula H2C═C(CH3)CO2(CH2CH2O)nCH3, MDL number MFCD00241432, PubChem Substance ID 24869402) and with end standing methyl group as in the formulation,

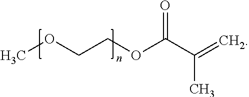

This M-PEG-M solution Mn 2,000 50 wt % in H2O was diluted in ethanol, having 50 w/w % of the M-PEG-M in H2O solution, 1 w/w % UV initiator and 49 w/w % ethanol. After the first UV cure of the OSTE+ microwell array, the OSTE+ array with free thiol and epoxy groups is completely submerged in the described ethanol solution with M-PEG-M Mn 2,000. Then, this is exposed to UV of 12 mW/cm² for 5 minutes. After this, the OSTE+ array is rinsed thoroughly first with ethanol and then with water. Next, the OSTE+ array is blow dried using an N₂ gun. The thermal cure of the OSTE+ array is performed in an oven at 60° C. overnight.

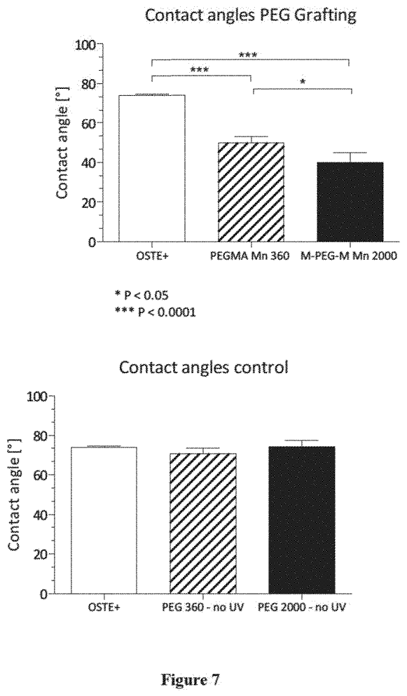

The successful grafting of the two types of PEG on the OSTE+ surface was validated using static contact angle measurements (FIG. 7). The contact angle is significantly lower for PEGMA Mn 360 grafted microwell arrays compared to OSTE+ arrays. Furthermore, the contact angle is significantly lower for M-PEG-M Mn 2,000 grafted microwell arrays compared to OSTE+ arrays and compared to PEGMA Mn 360 grafted microwell arrays (FIG. 7A). To validate that the decrease in contact angle is due to the chemical reaction between the PEG methacrylate and the free thiol and not due to physical adsorption, a control was included for each PEG. In this control, OSTE+ surfaces were simply submerged in the same PEG solutions, but without exposure to UV light. As seen in the FIG. 7B, no decrease in contact angle was observed for the controls, which confirms the specificity of the grafting process.

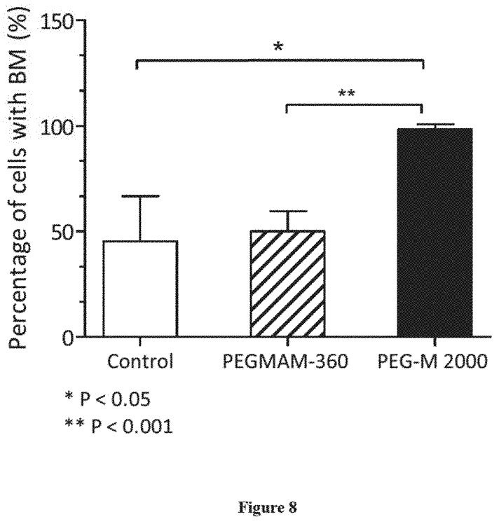

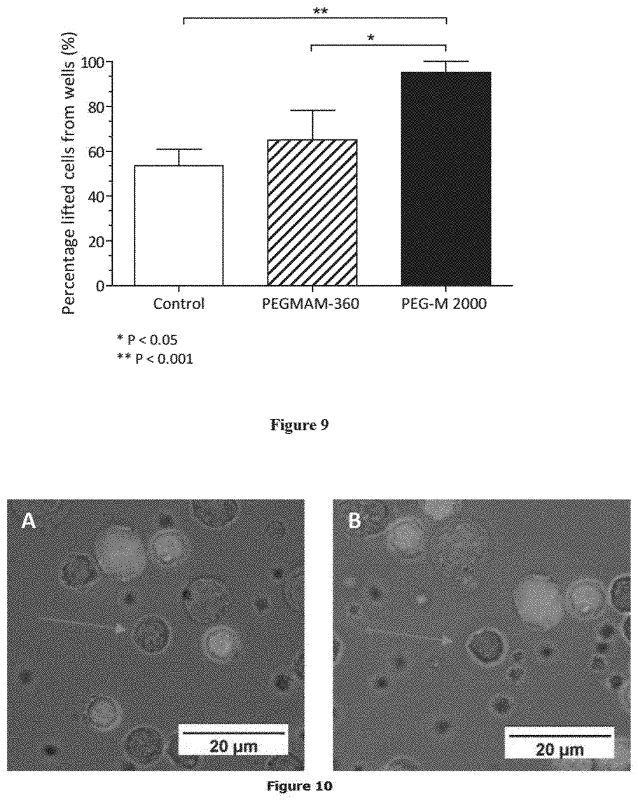

To study the effect of the PEG-grafting on the surface interactions between the cells and the microwells, the Brownian motion (BM) of the cells and the efficiency of the optical tweezers to lift cells out of the microwells were determined (FIG. 8 and FIG. 9, respectively). For BM study, B cells were seeded in 11±1 μm depth microwells and, after 2 hours incubation, imaged under the microscope at 100× objective. Microwell arrays without treatment (Control, OSTE+ arrays), with PEGMA-360 grafted and with M-PEG-M-2,000 grafted were tested and videos of at least 15 seconds were recorded for the different arrays. A random motion of cells in wells for more than 10 seconds was classified as “BM movement” (classified as 1); absence of movement was taken as “without BM movement” (classified as 0). This study gives an indication if the cells were in suspension or were adhered to surface of microwells and can be used as a prediction if the cells would be elevated by optical tweezers or not. These results show that M-PEG-M Mn 2,000 on the surface significantly increase Brownian motion (FIG. 8). Then, cells with Brownian motion were selected and it was attempted to optically tweeze the cell out of the microwell. The efficiency of B cells lifted from the microwell array was increased for arrays grafted with M-PEG-M Mn 2,000 as compared to OSTE+ microwells without PEG or with PEGMA Mn 360 (FIG. 9). PEGMA Mn 360 did not have the same desired effect as M-PEG-M Mn 2,000.

Upon the presence of 7-AAD, the viability of cells was evaluated before and after trapping with optical tweezer. It was observed that trapping a single viable cell for up to 10 mins did not compromised the cell membrane integrity (FIG. 10).

### Example 2—Microfluidic Channel for Controlled B Cell Seeding and B Cell Identification for Specific Antibodies

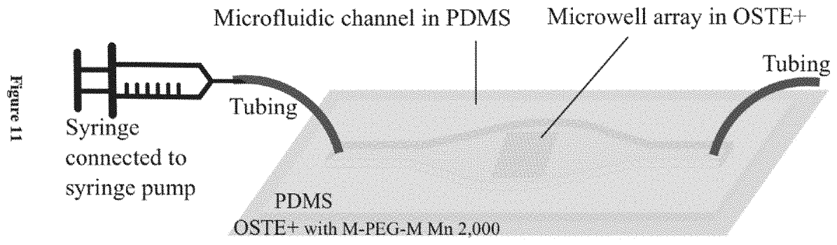

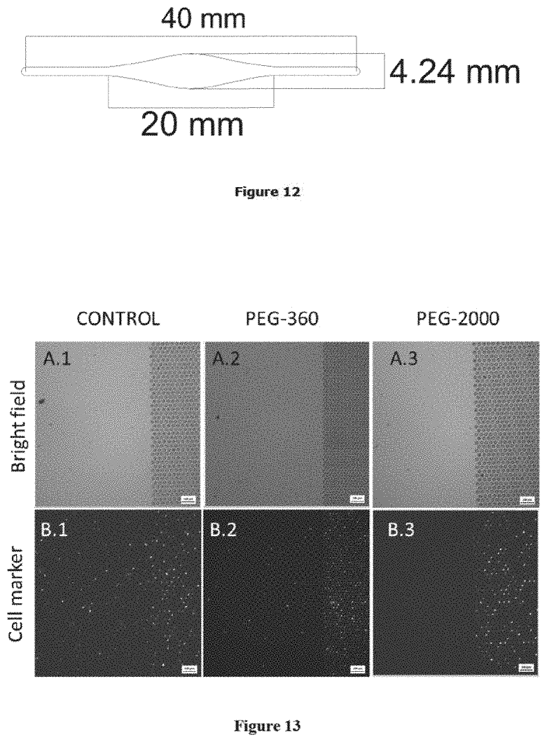

To have more control over the B cell seeding and B cell identification, we bonded a PDMS (Polydimethylsiloxane) microfluidic channel on top of the OSTE+ microwell array (FIG. 11 and FIG. 12). The PDMS (SYLGARD 184, 1.1 KG) was made in 10:1 ratio base:curing agent and cured for 5 hours at 60° C. in an oven. Two access holes were punched in the PDMS using a biopsy puncher of 1 mm diameter. Then, the PDMS was activated using oxygen plasma for 30 seconds at 200 mTorr and 30 W. The PDMS channel was placed over the OSTE+ microwell array that only had the first UV cure, not yet the thermal cure. The PDMS on OSTE+ was placed overnight in the oven at 60° C.

Similar grafting of PEG as described in example 1 of PEGMA Mn 360 and M-PEG-M Mn 2,000 was also performed in combination with the PDMS channel. Similar as example 1, the PEGMA Mn 360 was grafted onto the OSTE+ surface by submerging the first cured OSTE+ microwell array in the solution with PEGMA Mn 360, ethanol and UV initiator. After the 5 min UV exposure at 12 mW/cm², the microwell array was thoroughly rinsed with ethanol and water. Then, the PDMS channel was activated with oxygen plasma as described above, and the PDMS channel was placed on top of the microwell array with PEGMA Mn 360. Since PEGMA Mn 360 has endstanding hydroxyl groups, the activated PDMS binds efficiently to these PEGMA Mn 360 on the surface in an oven overnight at 60° C. Since M-PEG-M Mn 2,000 does not have an endstanding hydroxyl group, the PDMS channel has to be attached to the OSTE+ prior to the M-PEG-M Mn 2,000 grafting. Therefore, first the PDMS channel was activated and placed on the OSTE+ surface after its first UV cure. The PDMS channel was tightened to the OSTE+ surface using 4 foldback clamps, one on each side. The M-PEG-M Mn 2,000 solution with ethanol and UV initiator as described above was then pipetted through the access holes of the PDMS channel until the entire channel was filled with the solution. Then, this set-up was exposed to UV for 5 min at 12 mW/cm². Then, the channel was thoroughly rinsed by pipetting ethanol and water through the channel. Next, the construct with OSTE+, PDMS and the clamps was placed in an oven overnight at 60° C.

Microwells of 11±1 μm deep and 11±1 μm diameter were fabricated in OSTE+ and combined with the PDMS channel (FIG. 11). Syringe pumps (Nemesys) were used for flow-based seeding of single B cells in the microwells. The B cells at concentration of 107 cells/ml were fluxed at flow rate −5 μl/min. After 5 mins, the flow was stopped, and cells were incubated in the channel for 30 mins. As a washing step, Medium (RPMI+10% fetal calf serum) or PBS can be used to wash the array by flushing the solutions for 30 mins at flow rate of −5 μl/min.

Due to interaction forces between the B cells and the OSTE+ surface, it was difficult to wash away non-seeded B cells as they were sticking to the OSTE+ surface. Therefore, the washing efficiency was compared between OSTE+ surface and OSTE+ surface grafted with PEG. The efficiency of washing non-seeded B cells was evaluated using fluorescence microscopy (FIG. 13). In this figure, you can see the border of a microwell arrays without PEG (control), with PEGMA Mn 360 and M-PEG-M Mn 2,000. Images were captured under the microscope with bright field and fluorescence light source. For the non-treated OSTE+, cells were found next to the microwell array, after attempted washing for 30 mins at −5 μl/min. An improvement was observed with microwell array treated with PEG-360 however still not optimal. For the OSTE+ with M-PEG-M Mn 2,000, the area next to the array is very clean, meaning that all non-seeded cells are washed away, which is desirable. Therefore, the surface that was grafted with M-PEG-M Mn 2,000 clearly improved the washing of the non-seeded B cells compared to OSTE+ and OSTE+ grafted with PEGMA Mn 360.

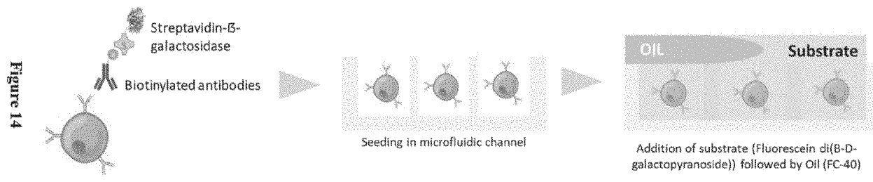

Using this microfluidic channel, the identification of specific B cells can be performed using an ELISA-based assay as illustrated in FIG. 14. By labelling a specific receptor on B cells surface (e.g., membrane bounded immunoglobulin) with biotinylated antibody, streptavidin with beta-galactosidase can be bounded. Upon the presence of a substrate (Fluorescein di(B-D-galactopyranoside), a fluorescent product is generated. B cells labelled against surface IgG were seeded in microfluidic channel and after adding 10 μg/ml of substrate for 1 min, the array was sealed using FC-40. In this scenario, individual wells are sealed and fluorescence can be detected beneath the presence of a positive cell. Preliminary results are shown in FIG. 15 but further optimization is contemplated. Later, an ELISA assay can be implemented to identify B cells expressing antibodies against a target protein.

### Example 3—Adapted PEG Grafting Enabling Both Efficient Optical Tweezing and Efficient Bonding of the PDMS Microfluidic Channel

Since the end standing methyl group of M-PEG-M Mn 2,000 complicates the bonding of a microfluidic PDMS channel with the grafted OSTE+ microwell array, as described in example 2, further investigation on another PEGMA was performed. A third type of PEG was used: Poly(ethylene glycol) methacrylate with average Mn 500 (Synonym: PEGMA, CAS Number 25736-86-1, Linear Formula H₂C═C(CH₃)CO(OCH₂CH₂)nOH, MDL number MFCD00081879 and with end standing hydroxyl group as in the formula

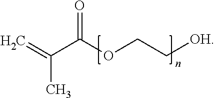

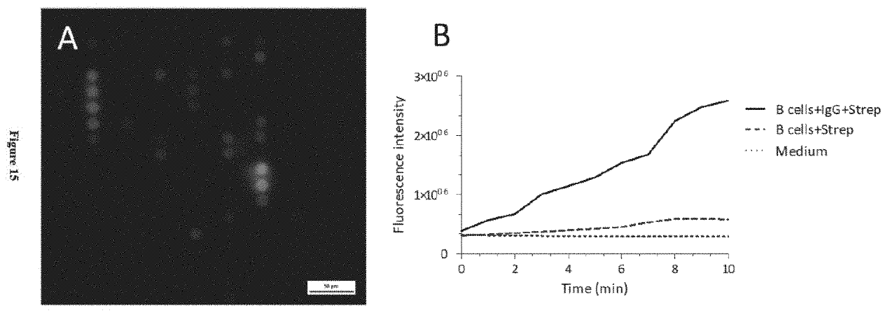

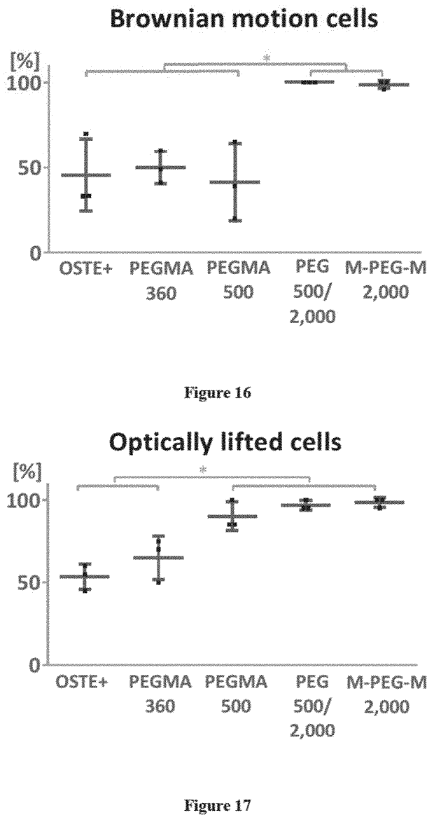

This PEGMA Mn 500 was diluted in ethanol, having 10 w/w % PEGMA, 2 w/w % UV initiator and 88 w/w % ethanol. After the first UV cure of the OSTE+ microwell array, the OSTE+ array with free thiol and epoxy groups is completely submerged in the described solution with PEGMA Mn 500. Then, this is exposed to UV of 12 mW/cm² for 5 minutes. After this, the OSTE+ array is rinsed thoroughly first with ethanol and then with water. Then, the same PDMS channel as in example 2 was activated with oxygen plasma as described above, and the PDMS channel was placed on top of the microwell array with PEGMA Mn 500. Since PEGMA Mn 500 has an end standing hydroxyl group, the activated PDMS binds efficiently to these PEGMA Mn 500 on the surface in an oven overnight at 60° C. A similar BM and optical tweezing study as in example 2 was carried out (FIG. 16 and FIG. 17). Grafting with PEGMA with Mn 500 was not sufficient to increase BM of cells compared to the non-grafted OSTE+ microwell array or the microwell array grafted with PEGMA Mn 360 (FIG. 16). However, optical tweezing efficiencies of cells with BM increased for PEGMA Mn 500 as compared to PEGMA Mn 360, which indicates that PEGMA Mn 500 reduced a specific cell adhesion more than PEGMA Mn 360 (FIG. 17).

To combine the low biofouling properties of M-PEG-M Mn 2,000 and the possibility of PDMS bonding with a surface grafted with PEGMA Mn 500, mixtures of these two PEG types were made. Several mixtures of different molar PEG ratios were tested as mentioned in Table 1. The corresponding weight percentages of the two PEG types, UV initiator and ethanol are mentioned in Table 1 as well. These mixtures were grafted to an OSTE+ microwell array by submerging the array in the solution and applying 5 min UV exposure at 12 mW/cm², after which the microwell array was thoroughly rinsed with ethanol and water. Then, a PDMS channel was activated with oxygen plasma as described above, and the PDMS channel was placed on top of the microwell array. It was found that for a PEGMA 500/M-PEG-M 2,000 ratio of 1 on 2 and 1 on 4, the PDMS could not bond to the grafted microwell array. For a PEGMA 500/M-PEG-M 2,000 ratio of 4 on 1, 2 on 1 and 1 on 1, the PDMS microfluidic channel could bond to the grafted microwell array. Since the equimolar ratio of 1 on 1 relatively contains most M-PEG-M 2,000 molecules, this ratio was selected for further experiments. This equimolar mixture will now be referred to as PEG 500/2,000.

As shown in the BM study, the same low biofouling performance was reached with microwell arrays grafted with PEG 500/2,000 as compared to arrays grafted with M-PEG-M 2,000 (FIG. 16). The optical tweezing study showed that 97±3% of the BM cells could be lifted out of the microwells using this surface chemistry (FIG. 17).

### Example 4—Single B Cell Transfer from the Array to the Outlet of the Microfluidic Chip

After the identification of the B cell with specific antibody and the optical tweezing of this cell out of the microwell, the cell still needs to be transferred to a reservoir such as a tube or a 96 well plate. In order to transfer a cell from the microwell array to a reservoir, the M-PEG-M Mn 2,000 or PEG 500/2,000 grafted array was integrated with a channel in PDMS as described in example 2 (FIG. 11).

This unidirectional flow for B cell seeding, washing and identification works well. However, a unidirectional channel is not sufficient to optically tweeze and collect only a single cell. The problem is that, for the tweezed cell to exit the channel, a continuous flow needs to be administered to the channel. The flow velocity needs to be high enough to transport the cell until the end of the channel. However, at this flow velocity, other cells also unwantedly pop out of the microwells and will thus also be transported until the end of the channel. This is contamination of unwanted cells which needs to be avoided.

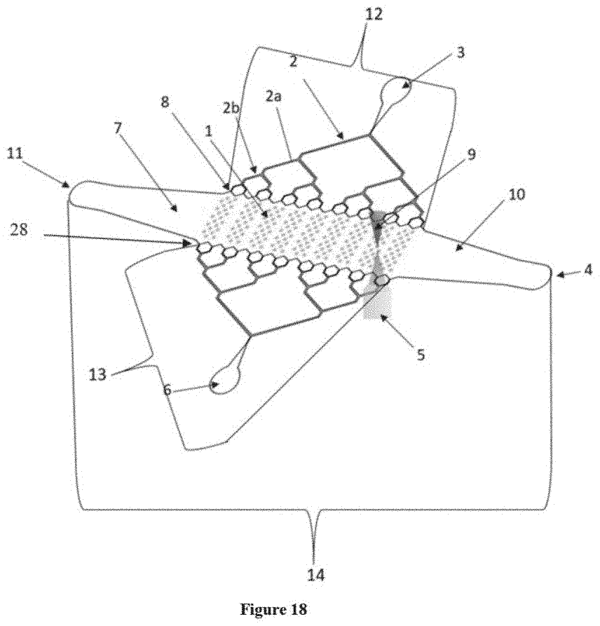

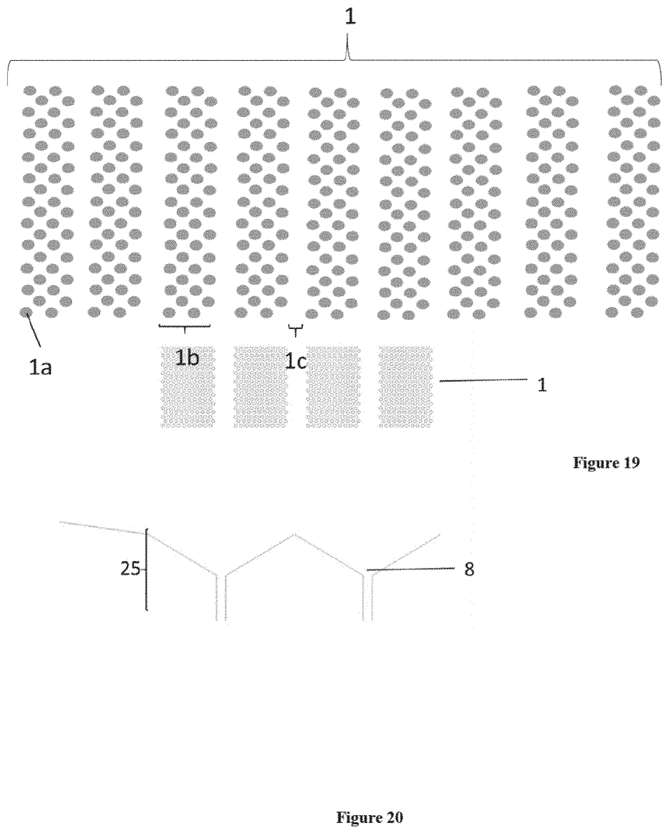

Therefore, a new design was used for the cell transfer with a bidirectional flow. As can be seen in FIG. 18, the microfluidic design consists of an oblong conduit [14] in which the microwell array [1] is positioned. As shown in FIG. 19, the microwell array [1] is designed in such a way that a series of microwell array groups are equally spaced from each other with a spacing that is at least double the size of a B cell (e.g., 30 μm). Each microwell columns are composed by microwells with size of 11±1 μm diameter and depth for single B cell seeding. The oblong conduit [14] consists of a fluid inlet port [11] for the delivery of cells and a fluid outlet port [4] for the waste collection (FIG. 18).

At both sides of the oblong conduit [14], a set of branched fluid channels [12,13] are positioned. The first set of branched fluid channels [12] is composed by one buffer inlet port [3] that splits multiple times into two fluid channels of the same width [2, 2a, 2b]. These fluid channels [2b] are connected to the oblong conduit [14] via a port [8] through a connection that is shaped like a funnel [25] (FIG. 20). The second set of branched fluid channels [13] is composed in the same manner. The two sets of branched fluid channels [12,13] are symmetrically positioned around the oblong conduit [14] and the microwell array [1] is positioned in between the two sets of fluid channels [12,13]. The sum of the cross-sections of every port [8] at the set of branched fluid channels [12] or [13] is 10-30 times smaller than the cross-section of the conduit above the microwell array [1] (FIG. 18).

Every inlet and outlet port of the conduit [14] is connected via microfluidic tubing to a syringe pump, pressure pump or peristaltic pump. The conduit [14] is produced in PDMS and is bonded to a microwell array in OSTE+ with M-PEG-M 2,000 or PEG 500/2,000 surface chemistry as described in example 1-3. The microfluidic chip is placed on a Nikon epifluorescence microscope equipped with an optical tweezers set-up as described in [1. Decrop, D. et al. Anal. Chem. 88, 8596-8603 (2016).].

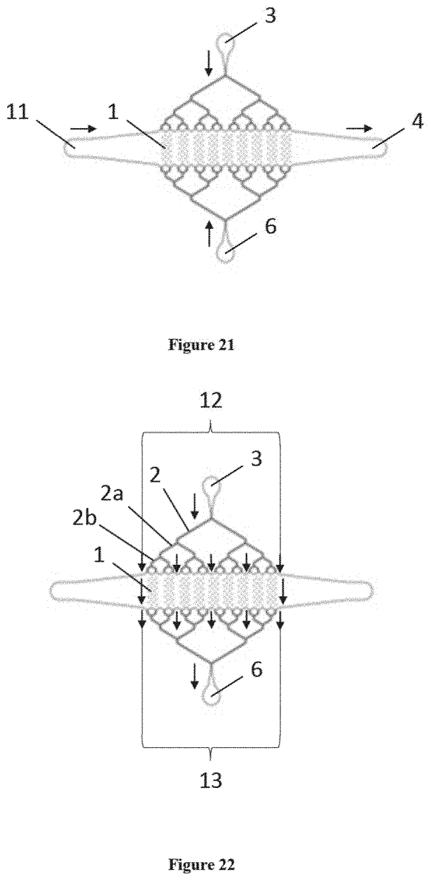

As a first step, illustrated in FIG. 21, single B cells are seeded in the microwell array [1] by operating buffer inlet port [11] and outlet port [4]. A reservoir, e.g., syringe or pipet tip, with B cells is connected to the inlet port [11] via microfluidic tubing and the cells are pushed into the conduit [14] using the pump. A buffer reservoir, e.g., syringe, is connected to the buffer inlet port [3] and buffer outlet port [6] that pushes buffer into the conduit [14], i.e., buffer outlet port [6] is now used as an inlet port. Because of the design of the fluid channels, the buffer will flow at the outer sides of the a port [8] and act as a sheath flow or curtain flow that focuses the cells over the microwell array [1] and prevents the cells from flowing into the two sets of branched fluid channels [12,13]. This ensures that unwanted cells do not flow into the branched fluid channels [12,13] and uniquely the desired cells are captured at the outlet port [6]. The buffer and non-seeded cells flow through the outlet port [4] via tubing to a waste reservoir (e.g., syringe). The design and connection to syringe pumps balances all flows, meaning that if e.g., 2 μL/min is pushed through cell inlet port [11] and 0.5 μL/min is pushed through the buffer inlet and outlet port [3,6], then liquid is removed through the outlet port [4] at a rate of 3 μL/min (=2+0.5+0.5 μL/min). After a certain short periods of flow, e.g., 2 minutes, all flows are stopped by stopping the syringe. Extra stability can be gained by including valves at the tubing that are closed manually or electronically when the flow needs to be stopped. When the fluid flows are stopped, cells can sediment into the microwells for a certain period of incubation, e.g., 5 minutes. Several cycles, e.g., 5, of cell delivery by the flows and cell sedimentation by stopping the flows can be performed to increase single cell seeding efficiency in the microwell [1].

After the cycles for cell delivery and seeding, B cells that are not seeded in the microwell array [1] need to be washed away, illustrated in FIG. 21. Washing buffer is pushed into the conduit [14] through buffer inlet port [11]. This can be performed by switching the syringe at inlet port [11] or by having a valve connected to the syringe that switches the cell suspension to buffer. Consequently, the non-seeded cells are pulled out of the chip through buffer outlet port [4]. A sheath flow from buffer inlet port [3] and buffer outlet port [6] is kept to avoid capture of undesired cell at the collection outlet. Again, all inlet and outlet flow rates are balanced.

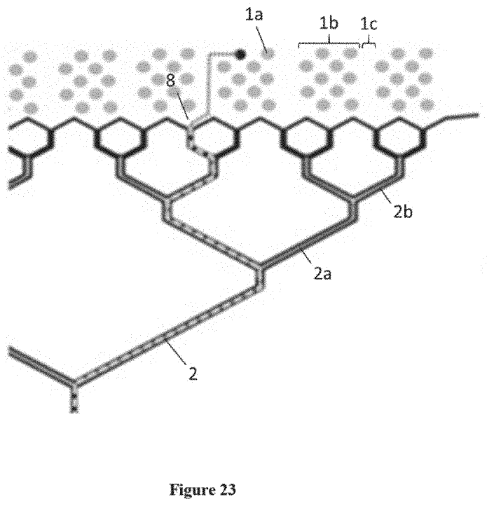

Once cells are seeded in the microwell array [1] and non-seeded cells are washed away, fluorescently-labeled B cells (e.g., antigen-specific B cells) can be identified using fluorescence microscopy as explained in example 2. Then, a desired single B cell needs to be retrieved from the microfluidic chip. For this, the optical tweezers [5],[9] are used and the flow is controlled by operating buffer inlet port [3] and buffer outlet port [6], as illustrated in FIG. 22. Buffer is pushed through buffer inlet port [3] and distributed through the first set of branched fluid channels [12], through the conduit over the microwell array [1] through the second set of branched fluid channels [13] towards the outlet port [6]. Because of the branched structure of the fluid channel sets [12] and [13], the flow speed is uniformly distributed over the microwell array [1]. Because the sum of the cross-sections of all the ports [8] is 10-30 times smaller than the cross-section of the conduit [14] above the microwell array [1], there is a 10-30 times drop in flow speed from the first set of fluid channels [12] to the conduit above the microwell array [1] and there is again a 10-30 times increase in flow speed from the conduit above the microwell array [1] to the second set of fluid channels [13]. Thanks to this feature, the flow speed above the microwell array is low enough so seeded cells are not disturbed and do not dislodge randomly from their microwell [1]. This low flow speed also allows single cell manipulation since the force exerted by the optical tweezers [5], [9] on the cell is larger than the forces exerted by the fluid flow. The microwell array [1] is specifically designed to minimize the exposure times of the focused beam laser of optical tweezers to the cell. Since the microwells are placed in microwell groups with spacing in between, the cell can be lifted out of the microwell by the optical tweezers [5,9], and transported to the nearest spacing. Then, the cell can be transported by the optical tweezers [5,9] towards the nearest port [8] of the second set of fluid channels [13]. Because the spacing in between the microwell groups is at least double the cell size (>20 μm), the lifting and transport of the cell from microwells arrays by optical tweezing to the nearest port [8] can be performed in several seconds (5-30 s). By tweezing the cell towards a port [8], the wall of the conduit [14] will be encountered. The presence of multiple ports and their specifically designed funnel shape [25] enables fast localization of the nearest port (1-5 s) and thus minimize interaction time of the optical tweezers with the cell. The cell is released at this port [8] by turning off the optical tweezers [5,9]. Because of the increase in flow speed in the second set of channels [13], the cell is transported by the flow towards the outlet for cell collection [6]. Because of the fluid transport, interference with the optical tweezers is not needed anymore, so interaction times with the optical tweezers are minimized, cell viability is maintained and cells can be retrieved within several seconds. The process of lifting and transporting the cell is illustrated in FIG. 23.

Because of the combined features of 1) The grouped microwell array [1b] with spacing [1c] and 2) The ports [8] that are designed as funnels for fast localization of the ports [8] and 3) The flow increase in the second set of branched channels [13], interaction time with the optical tweezers and the target cell are minimized and thus cell viability is maintained.

Because of the combined features of 1) The sheath flow preventing cells to enter in the branched fluid channels [12] and [13], 2) The uniform flow speed distribution above the microwell array [1], and 3) The 10-30 folds increase in flow speed in the second set of branched channels [13] allow to transport only the target cells to the outlet [6] without having any contamination from other unwanted cells.

The combination of all these features enables the selection of rare cells from a large population and collect the desired cells in a cell-by-cell manner with following advantages: 1) without contamination of other cells, 2) without losing cells in microfluidic tubing or in dead volumes in the microfluidic chip, 3) in collection volumes that are much lower than those achieved by other technologies.

### Example 5—Retrieval of Single B Cells from the Microfluidic Chip

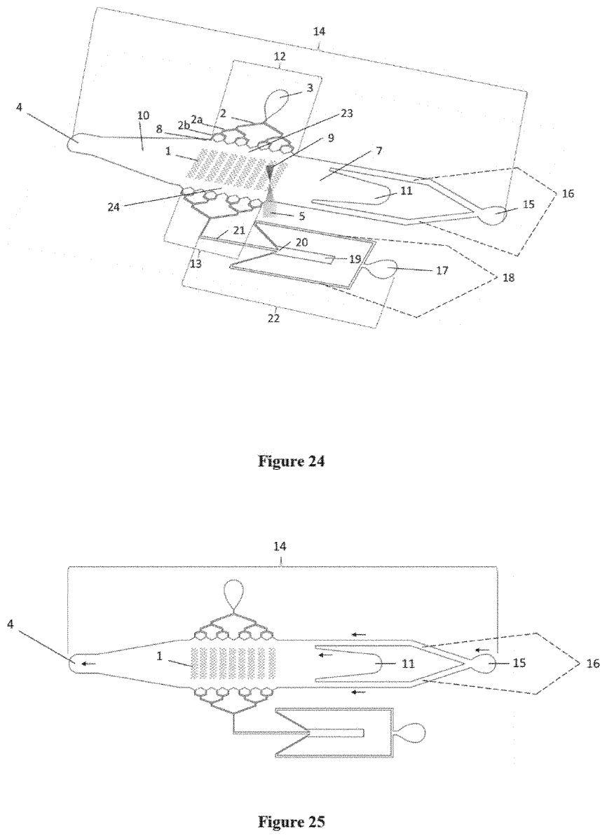

Additional features were added to the design explained in example 4 to on the one hand apply the sheath flow in a different way and on the other hand to retrieve single cells from the microfluidic chip to an off-chip reservoir for further analysis. This design can be found in FIG. 24.

The adapted microfluidic conduit [14] consists of an oblong conduit [14] in which the microwell array [1] is positioned (FIG. 24). The microwell array [1] (FIG. 19) is designed in such a way that a series of microwell array groups [1b] are equally spaced from each other with a spacing [1c] that is at least double the size (e.g., 30 μm) as the B cell. Each microwell group [1b] is composed by microwells [1a] with size of 11±1 μm diameter and depth for optimal single B cell seeding. The oblong conduit [14] consists of a cell inlet port [11] for the delivery of cells and a fluid outlet port [4] for the waste collection. The cell inlet port [11] can also be used as a fluid outlet port when needed. An extra buffer inlet port [15] is connected to two fluid channels [16] in which the buffer will be flowing at the outer sides of the oblong conduit [23], [24] towards the outlet port [4]. At both sides of the oblong conduit [14], a set of branched fluid channels [12,13] are positioned. The first set of branched fluid channels [12] starts from one buffer inlet port [3] and splits multiple times into two fluid channels with the same width [2, 2a, 2b]. These fluid channels [2b] are connected to the oblong conduit [14] via a port [8] through a connection that is shaped like a funnel [25] (FIG. 20). The second set of branched fluid channels [13] is composed in the same manner. The two sets of branched fluid channels [12,13] are symmetrically positioned around the oblong conduit [14] and the microwell array [1] is positioned in between the two sets of fluid channels [12,13]. The sum of the cross-sections of every port [8] at the set of branched fluid channels [12] or [13] is 10-30 times smaller than the cross-section of the conduit above the microwell array [1]. A space of >100 μm separates the microwell array [1] from the two sets of branched fluid channels [12,13] as indicated by region [23] and [24]. The second set of fluid channels [13] is connected to a droplet generation geometry [22]. In the latter, an oil inlet port [17] is connected to two oil fluid channels [18] that intersect with the fluid channel before droplet generation geometry [21] which is an elongation of the set of fluid channels [13]. Buffer flowing from the buffer inlet [3] through the first set of fluid channels [12] over the microwell array [1] through the second set of fluid channels [13] to the channel before droplet generation geometry [21] will contact the oil coming from oil fluid channels [18] at the position of droplet generation [20] to form buffer-in-oil droplets. These formed buffer-in-oil droplets will flow to the outlet port [19] in which tubing is connected to an off-chip reservoir. The width of the fluid channel at the outlet port [19] is the same (±25 μm) as the inner diameter of the microfluidic tubing.

Every inlet and outlet port of the conduit [14] is connected via microfluidic tubing to a syringe pump, pressure pump or peristaltic pump. The conduit [14] is produced in PDMS and is bonded to a microwell array in OSTE+ with M-PEG-M 2,000 or PEG 500/2,000 surface chemistry as described in example 1-3. The microfluidic chip is placed on a Nikon epifluorescence microscope equipped with an optical tweezers set-up as described in [1. Decrop, D. et al. Anal. Chem. 88, 8596-8603 (2016).].

As a first step, single B cells are seeded in the microwell array [1] by operating buffer inlet port [15], cell inlet port [11] and outlet port [4], as illustrated in FIG. 25. A reservoir, e.g., syringe or pipet tip, with cells is connected to the cell inlet port [11] via microfluidic tubing and the cells are pushed into the conduit [14] using the pump. A buffer reservoir, e.g., syringe, is connected to the buffer inlet port [15] and buffer is pushed into the conduit [14]. Because of the design of the fluid channels [16], the buffer will flow at the outer sides of the conduit [23], [24] and act as a sheath flow or curtain flow that focuses the cells over the microwell array [1] and prevents the cells from flowing into the two sets of branched fluid channels [12,13]. This prevents unwanted, random B cells from flowing in the branched fluid channels and causing contamination in the second set of branched fluid channels and the droplet generation geometry. The buffer and non-seeded cells flow through the outlet port [4] via tubing to a waste reservoir (e.g., syringe). The design and connection to syringe pumps enables all flows to be balanced meaning that if for example 2 μL/min is pushed through cell inlet port [11] and 1 μL/min is pushed through the buffer inlet port [15], then liquid is removed through the outlet port [4] at a rate of 3 μL/min. After a certain period of flow, e.g., 2 minutes, all flows are stopped by stopping the syringes. Extra flow stability can be gained by including valves at the tubing that are closed manually or electronically when the flow needs to be stopped. When the fluid flows are stopped, cells can sediment into the microwells [1a] for a certain period of time, for example 5 minutes. Several cycles, e.g., 5, of cell delivery by the flows and cell sedimentation by stopping the flows can be performed to increase single cell seeding efficiency in the microwell [1].

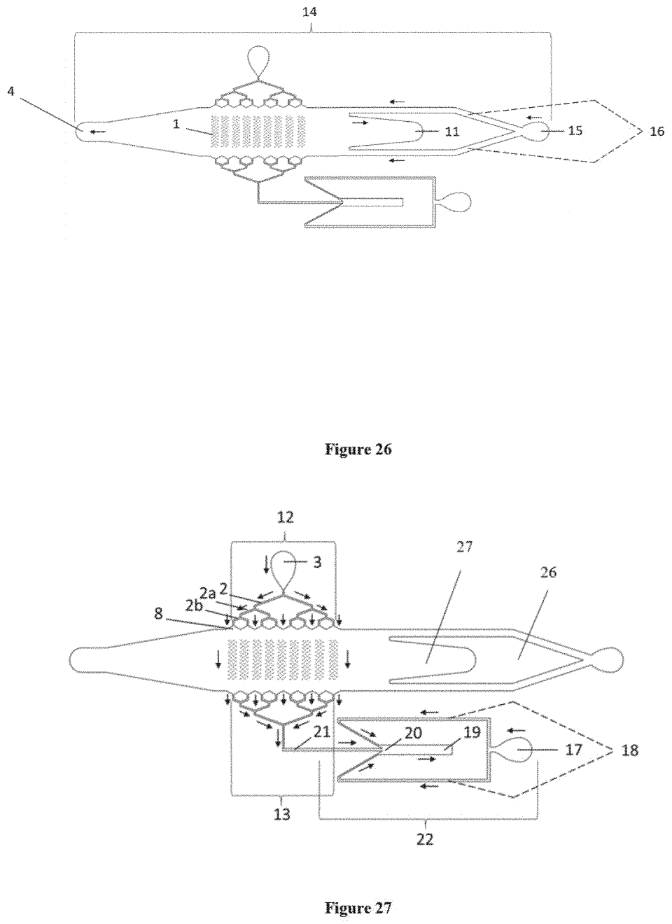

After the cycles for cell delivery and seeding, B cells that are not seeded in the microwell array [1] need to be washed away, as illustrated in FIG. 26. This can be performed by switching on the flows at the inlet port [15] and [11] and outlet port [4]. Washing buffer is pushed into the conduit [14] through buffer inlet port [15]. The buffer and non-seeded cells are pulled out of the chip through buffer outlet port [4] and by pulling through the cell inlet port [11]. The cell inlet port [11] is now thus used as an outlet. This is done because it was observed that cells sediment below the tubing of inlet port [11] during cell seeding, so if the inlet port [11] was used as an inlet for pushing in washing buffer, the sedimented cells were gradually taken along with the flow and cells were constantly delivered to the region of the microwell array [1]. Since this inhibited washing of all non-seeded cells, the washing buffer was pulled into port [11] during washing. Again all inlet and outlet flow rates are balanced.

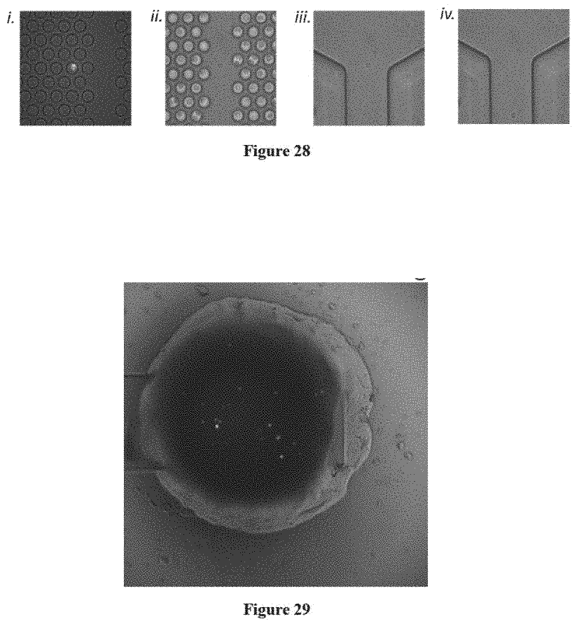

Once cells are seeded in the microwell array [1] and non-seeded cells are washed away, fluorescently labeled B cells (e.g., antigen-specific B cells) can be identified using fluorescence microscopy as explained in example 2. Then, a B cell of interest needs to be retrieved from the microfluidic chip. For this, the optical tweezers [5],[9] are used and the flow is controlled by operating buffer inlet port [3], oil inlet port [17] and the outlet port for buffer-in-oil droplets [19], as illustrated in FIG. 27. Buffer is pushed through buffer inlet port [3] and distributed through the first set of branched fluid channels [12], through the conduit over the microwell array [1] through the second set of branched fluid channels [13] towards the outlet for buffer-in-oil droplets [19]. Because of the branched structure of the fluid channel sets [12] and [13], the flow speed is uniformly distributed over the microwell array [1]. Because the sum of the cross-sections of all the ports [8] is 10-30 times smaller than the cross-section of the conduit [14] above the microwell array [1], there is a 10-30 times drop in flow speed from the first set of fluid channels [12] to the conduit above the microwell array [1] and there is again a 10-30 times increase in flow speed from the conduit above the microwell array [1] to the second set of fluid channels [13]. Thanks to this feature, the flow speed above the microwell array is selected low enough so seeded cells are not disturbed and do not dislodge randomly from their microwell [1], preventing contamination with unwanted cells. This low flow speed also allows the single cells manipulation since the force exerted by the optical tweezers [5], [9] on the cell is larger than the forces exerted by the fluid flow. The microwell array [1] is specifically designed to minimize interaction times of the optical tweezers with the cell. Since the microwells [1a] are placed in microwell groups [1b] with spacing [1c] in between, the cell can be lifted out of the microwell [1a] by the optical tweezers [5],[9], and transported to the nearest spacing [1c]. Then, the cell can be transported by the optical tweezers [5,9] towards the nearest port [8] of the second set of fluid channels [13]. Because the spacing [1c] in between the microwell groups [1b] is at least double the cell size (>20 μm), the transport of the cell by optical tweezing to the port [8] can be performed in several seconds (5-30 s) since there is no hindrance of movement by other cells or microwells or cells in microwells. By tweezing the cell towards a port [8], the wall of the conduit [14] will be encountered. The presence of multiple ports and their specifically designed funnel shape [25] enables fast localization of the nearest port (1-5 s) and thus minimize interaction time of the optical tweezers with the cell. The cell is released at the nearest port [8] by turning off the optical tweezers [5,9]. Because of the increase in flow speed in the second set of channels [13], the cell is transported by the flow towards the droplet generation geometry [22]. Because of the fluid transport, interference with the optical tweezers is not needed anymore, so interaction times with the optical tweezers are minimized, cell viability is maintained and cells can be retrieved within several seconds. Microscopy images of the tweezing process can be found in FIG. 28.

Once the desired B cell is optically tweezed out of the microwell and transported by the flow in the into the droplet generation geometry [22], the cell needs to be transferred from the chip to an off-chip reservoir such as a tube, 96 well plate or other, for further downstream analysis. Conventionally, holes are punched in the PDMS for connecting the chip via tubing to a pump or reservoir [2. Wang, X. et al. Lab Chip 11, 3656 (2011)]. This tubing is inserted via the top of the PDMS microfluidic channel. Using this standard set-up, it was observed that the isolated B cells sediment below the lumen of the tubing and consequently do not enter the tubing (FIG. 29), even though there is a continuous microfluidic flow. Consequently, using this traditional set-up, cells are residing in dead volumes in the chip and cannot be transported to an off-chip reservoir. Moreover, if a cell would enter in the tubing, the cell can still be lost in the tubing because of sedimentation in the tubing or adhesion to the tubing wall. As an alternative, in literature, cells were retrieved from microfluidic sorting chips by manually pipetting the cells out of an opening in the chip using a micropipette [3. Kovac, J. R. & Voldman, J. Anal. Chem. 79, 9321-9330 (2007).]. However, such a pipetting step complicates automation and requires manual intervention with the microfluidic chip, which caused random dislodging of B cells from microwells in the case of this invention. Moreover, it cannot be ensured that all cells are effectively collected from the chip by pipetting, since cells might adhere to the bottom of the chip or not all liquid is effectively pipetted.

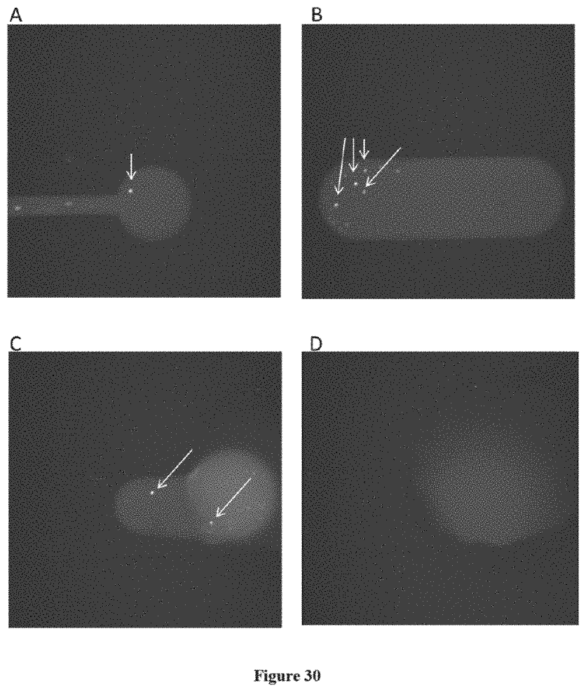

To enable single cell retrieval in automated and efficient manner through a tubing, a droplet generation geometry [22] was implemented in the microfluidic design (FIG. 24). Herein, the flow in the second set of branched fluid channels [13] that carries the tweezed cell towards the outlet, is pinched off into droplets by the oil at [20] (Flow scheme in FIG. 27). Similar to droplet microfluidics, the oil acts as the continuous phase or the carrier phase, in which the buffer is dispersed [4. Joensson, H. N. & Andersson Svahn, H. Angew. Chemie—Int. Ed. 51, 12176-12192 (2012).]. The buffer droplets are mostly empty, but will contain a single cell when a desired B cell is tweezed to a port [8]. The oil carrier phase then pushes the droplets into the tubing connected to the outlet port [19]. When the droplet contains a cell, this cell is also pushed into the tubing connected to the outlet port [19] and can be transported to an off-chip reservoir. To illustrate this, FIG. 30 shows how a droplet containing multiple B cells and platelets is pushed into the tubing by the oil carrier phase. The outlet of this tubing is then positioned in or above a reservoir, such as a tube or 96-well plate, to capture the generated droplets. To push the droplets into the microfluidic tubing, it is important that the width of the fluid channel at the outlet port [19] is the same (±25 μm) as the inner diameter of the microfluidic tubing. Therefore, the generation of droplets allows the retrieval of desired single cells in a cell-by-cell manner without losing cells in dead volumes in the fluid channels or at the outlet port [19]. Moreover, since the droplet volume is in between 0.01 μL and 0.05 μL, this feature allows the collection of very low volumes which is not possible by pipetting or by buffer flows in tubing. This feature makes the current invention suitable for single cell retrieval for further single cell analysis approaches (e.g., single cell sequencing) at which reduced volumes are required.

Because of the combined features of 1) The grouped microwell array [1b] with spacing [1c] and 2) The multiple ports [8] that are designed as funnels for fast localization of the ports [8] and 3) The flow increase in the second set of branched channels [13], interaction time with the optical tweezers and the target cell are minimized and thus cell viability is maintained. In addition, because of the combined features of 1) The sheath flow preventing cells to enter in the branched fluid channels [12] and [13], 2) The uniform flow speed distribution above the microwell array [1], and 3) The 10-30 folds increase in flow speed in the second set of branched channels [13], only transport of the target cells to the outlet [19] is allowed, without having any contamination from other unwanted cells. Because of the droplet generation geometry, the target cell can be transported from the conduit [14] via a tubing to an off-chip reservoir for further analysis. These collected volumes are small (0.01 to 0.5 μL) so cellular RNA is hardly diluted, which is necessary for RNA sequencing.

The combination of all these features enables the selection of very rare cells from a large population of cells and the collection of the target cells in a cell-by-cell manner without contamination of other cells and without losing cells in microfluidic tubing or in dead volumes in the microfluidic chip, and in collection volumes that are much lower than those achieved by other technologies.

Based on the flow rates used in the conduit at inlet port [3] and oil inlet port [17], and the diameter of the tubing, the time for the droplet to arrive at the end of the collection tubing can be determined. Then, during the correct time interval, the droplets can be captured in the off-chip reservoir. This reservoir can be a tube, 96 well plate, or other. This reservoir will then contain a number of empty droplets and one droplet containing the target cell. For further analysis, such as single cell RNA sequencing, the cell needs to be brought in contact with reagents, such as lysis buffer and PCR reagents, for which the droplets can be merged using for example a chemical such as perfluorooctanol and chloroform, or an antistatic gun [5. Karbaschi, M., Shahi, P. & Abate, A. R., Biomicrofluidics 11, (2017)]).

The oil used for droplet generation in these experiments was QX200™ Droplet Generation Oil for EvaGreen from Bio Rad, but other oil with surfactants can be applied for generation of stable water-in-oil droplets.

The geometry for the droplet generation [22] used here is drawn in FIG. 24, but can be any other design with which droplets can be generated, such as a T-junction or a flow focusing junction [4. Joensson, H. N. & Andersson Svahn, H. Angew. Chemie—Int. Ed. 51, 12176-12192 (2012).].

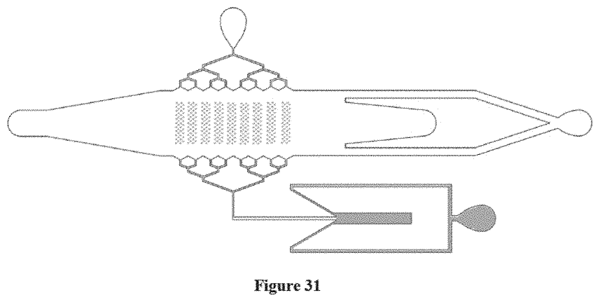

To produce stable buffer-in-oil droplets, the surface of the droplet generation module had to be hydrophobic. Since the PEG grafted surface is hydrophilic, a hydrophobic treatment had to be applied on this part of the design, as indicated in FIG. 31. This was done by flushing an Aquapel solution or a solution of 1% Trichloro(1H,1H,2H,2H-perfluorooctyl)silane in HFE 7500 through the chip and incubating the chip for 30 min in an oven at 65° C.

Thus, the claims following the detailed description are hereby expressly incorporated into this detailed description, with each claim standing on its own as a separate embodiment of this invention.

1.-40. (canceled)

41. A cell sorting device comprising:
a conduit, which is elongate or oblong, and which has an upstream portion having at least one inlet port for the conduit and a downstream portion having at least one outlet port for the conduit,
a microarray of microwells positioned within the conduit between the upstream and downstream portions, wherein the microarray is further positioned in the conduit between ports or apertures of a first set of branching channels and ports or apertures of a second set of branching channels, and
wherein the first and second sets of branching channels are not contained within the conduit, but are in fluid communication with the conduit's interior via ports or apertures in the conduit.

42. The cell sorting device of claim 41, wherein the microwells are arranged in parallel arrays of microwells or in rows of microwells, which parallel arrays or rows are separated by parallel partitions or spaces, and which parallel arrays or rows are positioned longitudinally between the ports or apertures of the first set of branching channels and the ports or apertures of the second set of branching channels.

43. The cell sorting device of claim 41, wherein a space exists between the microarray and zones of the conduit's wall where the branching channels are in fluid communication with the conduit via the ports or apertures.

44. The cell sorting device of claim 43, wherein the upstream portion comprises a first fluid inlet port more distal from the microarray and a second fluid inlet port relatively more proximate to the microarray,
wherein the first fluid inlet port is in fluid communication with two fluid channels which each extend laterally and longitudinally with a space and open proximate the space so as to create, when operational, a lateral flow of a sheath fluid that sandwiches a core fluid, and
wherein the second fluid inlet port opens more in the upstream portion's core so that, when operational, a core fluid stream is created directed towards the microarray.

45. The cell sorting device of claim 41, further comprising:
a solid object positioned in the upstream portion of the conduit with a space between the solid object's rim and part of the conduit's wall so as to form channels extending from the first fluid inlet port, and
wherein the second fluid inlet port opens in a cavity formed by a recess in an edge of the solid object, which recess faces the microarray so that, when operational, a core fluid stream with cells releases into the cavity and towards the microarray and by lateral flow of a sheath fluid that sandwiches a core fluid directed towards the microarray.

46. The cell sorting device of claim 45, wherein the solid object is a Y-shaped solid plate.

47. The cell sorting device of claim 41, wherein the microarray is a microwell array plate that, at its edge, is aligned between the first and second sets of branching channels and between two opposing fluid conduit ports.

48. The cell sorting device of claim 41, wherein an optical tweezer is positioned under the microarray's plane or bottom.

49. The cell sorting device of claim 41, wherein the microarray is for single cell per microwell trapping and lifting of viable cells, and wherein the microwells are in a matrix having a thiol polymer with methoxy polyethylene glycol methacrylate chains at the surface, wherein the chains have a number average molecular weight or an Mn value in a range of between 1,500 and 2,500 g/mol.

50. The cell sorting device of claim 49, wherein the chains have a number average molecular weight or an Mn value in a range of between 1,900 and 2,100 g/mol.

51. The cell sorting device of claim 49, wherein the chains have a number average molecular weight or an Mn value of 2,000 g/mol.

52. The cell sorting device of claim 41, wherein the microarray is for single cell per microwell trapping and lifting of viable human B cells, and wherein the microwells are in a matrix having a thiol polymer with methoxy polyethylene glycol methacrylate chains at the surface, wherein the chains have a number average molecular weight or an Mn value in a range of between 1,500 and 2,500 g/mol.

53. The cell sorting device of claim 52, wherein the chains have a number average molecular weight or an Mn value in a range of between 1,900 and 2,100 g/mol.

54. The cell sorting device of claim 41, wherein the microwells have a diameter in a range of between 9-14 μm.

55. The cell sorting device of claim 54, wherein the microwells have a diameter in a range of between 10-13 μm.

56. The cell sorting device of claim 49, wherein the thiol polymer is a thiol/ene polymer.

57. The cell sorting device of claim 41, wherein the microwells are in a matrix having a thiol polymer with methoxy polyethylene glycol methacrylate chains and oxiranyl groups at the surface.

58. The cell sorting device of claim 41, wherein the microwells are in a matrix comprising a thiol-ene polymer of the group consisting of off-stoichiometry thiol-enes polymer and off-stoichiometry thiol-ene-epoxies polymer.

59. The cell sorting device of claim 41, further comprising apparatus for trapping a single viable human B cell in a well and selectively lifting a single viable human B cell from the well without affecting the cell's viability, wherein the apparatus comprises a single beam optical tweezer having a 900-1200 nm range wavelength and a laser power of between 400 mW and 600 mW, and further wherein the microarray forms part of the apparatus.

60. A method of manipulating a single viable cell so as to preserve the cell's viability, trapping a single cell in single well, analyzing a selected single cell, identifying a B cell expressing a selected protein, and/or optically trapping and lifting a selected cell by an optical tweezer for further manipulating the cell, the method comprising:
using the cell sorting device of claim 41 in the method.

61. A method of cell seeding, washing of non-seeded cells, and delivery of reagents for identification of a cell, the method comprising:
using the cell sorting device of claim 41 in the method.
